(19) United States
(12) Patent Application Publication
(10) Pub. No.: US 2023/0393155 A1
(43) Pub. Date: Dec. 7, 2023

(54) LATERAL FLOW IMMUNOASSAY DEVICES WITH DYNAMIC TRACKING AND METHODS OF USING THE SAME

(71) Applicant: Quanovate Tech Inc., San Ramon, CA (US)

(72) Inventors: Ning LI, Hangzhou (CN); Yongjie SHI, Hangzhou (CN); Jiacheng ZHANG, Hangzhou (CN); Katerina SHKODZIK, Minsk (BY); Sylvia M. KANG, Castro Valley, CA (US); Zheng YANG, Castro Valley, CA (US)

(21) Appl. No.: 18/328,680

(22) Filed: Jun. 2, 2023

Related U.S. Application Data
(60) Provisional application No. 63/348,356, filed on Jun. 2, 2022.

(51) Int. Cl. G01N 33/76; G01N 33/543

(52) U.S. Cl. CPC G01N 33/76; G01N 2333/59; G01N 33/54388

(57) ABSTRACT

Systems and methods for detecting an analyte include test wands configured to employ immunochromatographic assay with new fluorescent labeling technology, a reading device that can specifically identify signals from wands and sync information to the mobile devices. The systems may quantitatively detect progesterone metabolites in urine to track the level changes, confirm ovulation, and/or evaluate menstruation status. The systems and methods assess and track an individual's ovulation cycle by monitoring a hormone pattern of that specific individual, and establishing a dynamic threshold as a baseline for changes in the specific subject's hormones.

### FIELD OF THE INVENTION

The present invention relates to an apparatus and method for detecting the presence or absence of analytes in a biological sample to assess a condition or status of a subject. In particular, the present invention is directed to lateral flow immunoassay devices for use in assessing the fertility of a female subject.

### BACKGROUND OF THE INVENTION

Lateral flow assay devices may provide rapid and cost-effective mechanisms for detecting the presence or absence of analytes in a sample. Lateral flow assay devices have been used to assess the ovulation or menstrual cycle of a subject, conventionally, by detecting progesterone metabolites in urine by calculating a level of pregnanediol glucuronide (PdG) and evaluating the calculated level relative to a predetermined PDG threshold level. Changes in the calculated level of pregnanediol glucuronide may be observed, for example, based on differences in color or color intensity of a detectable label relative to a reference color, or based on a simple image, such as the display of one or more lines. Examples of such conventional methods are disclosed by Ecochard, R., et al. “Use of urinary pregnanediol 3-glucuronide to confirm ovulation.” Steroids 78.10 (2013): 1035-1040; and Koczula, K. M., and A. Gallotta. “assays Lflow.” Lateral flow assays. Essays Biochem 60 (2016): 111-20.

More recently, Beckley (U.S. Pat. Nos. 11,029,321 and 11,061,026) has taught the use of a lateral flow assay comprising both a sandwich assay and a competitive assay to detect the presence or absence of at least PdG in a non-serum bodily fluid. The lateral flow assay taught in this instance includes a first conjugate pad saturated with monoclonal anti-pregnanediol glucuronide (anti-PdG) antibodies conjugated to a first visual label in a concentration of 1-10 μg/mL; a second conjugate pad saturated with selected antibodies chosen from anti-luteinizing hormone, anti-follicle stimulating hormone, and anti-human chorionic gonadotropin, and conjugated to a second visual label; and a membrane comprising a first testing zone and a second testing zone. The membrane provides a first observable result for the absence or presence of PdG at or above a PdG threshold of 3-20 μg/ml, as indicated by a color display at the first visual label in the first testing zone, and provides a second observable result for the absence or presence of the selected hormone(s), as indicated by a color display at the second visual label in the second testing zone.

Despite the advances provided to date in the art, there remains a number of shortcomings in the current state of the art. For example, conventional approaches provide only a means for analyzing a hormone metabolite at a single moment in time, without means for tracking a pattern of test results. The conventional systems and methods are also subject to analysis errors due to inappropriate selection of antibodies and type of carrier proteins, and the test results are subject to misinterpretation due to the use of results displays that may be easily misread by a user (e.g., a number of lines or variances in color).

Thus, there remains a need in the art for immunoassay devices that can provide superior test results to a user while also reducing the likelihood of misinterpretation by the user.

### SUMMARY OF THE INVENTION

The present invention is inclusive of a system including test wands configured to employ immunochromatographic assay with new fluorescent labeling technology, a reading device that can specifically identify signals from wands and sync information to the mobile devices, and application software. Systems according to the present invention may quantitatively detect progesterone metabolites in urine to track the level changes, confirm ovulation, and/or evaluate menstruation status.

Systems according to the present invention include a label that facilitates quantitative testing. Preferably, the label is a fluorescent dye conjugated with antibodies by preparatory methods. The fluorescent dyes have specific excitation and emission. Under the irradiation of a specific wavelength of the light source, that remains consistent with the fluorescent dye, the fluorescent dye emits light with a wavelength slightly different from that of the light source. Under certain conditions, the concentration of the fluorescent material is proportional to the fluorescent intensity. The labeled antibody solution contains antibodies ranging from about 0.01 mg/ml to about lmg/ml, and the fluorescent ranges from about 0.01 mg/ml to about 0.1 mg/ml. The volume of the labeled antibodies solution is from about 0.1 μl to about 10 μl.

The analyzer contains a light source configured specifically to excite the fluorescent dye and a receiving device configured to receive an emission signal from the excited fluorescent dye. The analyzer estimates the concentration of the analyte by measuring a fluorescence intensity of the excited fluorescent dye. See, for example, FIGS. 8-10B. When the detection module scans the entire wand, the detector receives excited fluorescent light at different positions during the scanning process. A scan pattern and fluorescent strength at various positions within the pattern may be obtained from this scanning. Based on the scan pattern, peak intensity or peak height/area/size of a signal waveform may be calculated. The status the wand and/or concentration of an analyte in a biological sample loaded on the wand may be further analyzed, with the further assistance of standard curve information and additional parameters that are stored on a communication and storage device (e.g., a microchip) embedded in the test wand, which may be further complemented by information stored in a memory on the analyzer.

The microchips embedded in the wands contain information about the type of the wands, expiration date, a standard curve for concentration calculation, etc. The information in the microchip is read into the analyzer once the test wand is inserted into the analyzer. The analyzer can distinguish the type of test wand based on the information from the microchip, so the analyzer can load the corresponding algorithm to interpret the fluorescent scan pattern and finally report the concentrations of hormones. The analyzer can read different types of wands and give corresponding concentrations of each hormone, e.g., Pregnanediol-3-Glucuronide(PdG). Each type of test wand can be used to test one or multiple analytes.

The present invention is further inclusive of an application for evaluating the immunoassay test results from a test wand without reliance on a fixed threshold, but instead through use of a dynamic threshold or tracking that changes based on the specific subject (e.g., a human female) that is being tested. Generally, urinary hormones vary from woman to woman, and from cycle to cycle, due to individual differences and hormone changes that different individuals may experience. As a result, a fixed threshold isn't suitable for every woman. The systems and methods of the present invention provide an improved assessment and tracking of an individual's ovulation cycle by monitoring a hormone pattern of that specific individual, rather than relying on a predetermined threshold as a default absolute value among the population of users.

A dynamic threshold is determined by counting multiple dimensions of PdG data, such as PdG values, and calculating the latest PdG gradient by setting sliding windows, time of test, and PdG data of the previous period, to build a model based on machine learning. This provides a much more accurate and reliable baseline for a specific subject beyond that which would otherwise be available from relying on only a single dimension of data in the form of a threshold value alone.

Dynamic models according to the present invention will facilitate more accurate predictions as to a subject's probability of ovulation, as well as a period of time before ovulation is most likely. Generally, devices and methods according to the present invention confirm ovulation by using average levels observed for the specific subject before ovulation as a baseline and then, when there is an increase in PdG reading for three consecutive days, and each day the measured PdG reading increases multiplicatively relative to the baseline, it can confirm ovulation.

Test wands according to the present invention comprise a sampling end for collecting a biological sample, a conjugate pad saturated with monoclonal anti-progesterone metabolites or other antibodies conjugated to a fluorescent label, a membrane comprising a testing zone and a control zone, an absorbent material for promoting chromatography, and a chip containing information of the wand and which is recognized by the analyzer.

An analyzer according to the present invention is a palm-sized portable device that performs the test based on fluorescence scanning technology. The analyzer comprises a wand insertion port for insertion of a test wand, a chip reading device for automatically identifying test wand information stored on a chip in the test wand, a scanning module for scanning the test signal of one or more reactions on the membrane of the test strip in a test wand, a display screen for displaying test results, and one or more communications modules (e.g., Bluetooth®) for synchronizing test results to one or more remote devices (e.g., a user's mobile computing device; a physician's monitoring device). The analyzer may recognize different test wands that are each configured for detect different substances.

Application software according to the present invention can be installed on both the analyzer and a user's mobile computing device. The application software generates test results taking into accounting PdG values generated from several successive tests. The test results may be transmitted from an application loaded at the analyzer to an application loaded at the user's personal computing device, allowing users to record and track their personal cycle and other related information, including a fertility score that evaluates a fertility status of the subject's cycle. An algorithm based on the cycle information and test data helps the analyzer learn the cycle for a specific user/subject.

The application software loaded on a user's personal computing device may provide test reminders, data storage, curve drawing, and fertility status evaluation. Test result history may also be stored and viewable through the application. The application may plot hormone curves based on a number of days tested. The application may also allow a user to edit information on their personal at any time.

In operation, a user will load a biological sample onto the test wand and will insert the test wand into the analyzer. The analyzer recognizes the wand by reading the chip information and starts to countdown. After a certain incubation time (coded in the microchip), the analyzer scans the test wand and obtains the test results by translating the intensity of a fluorescent signal to the concentration of the progesterone metabolites via information pre-stored on the chip of the wand and/or a memory of the analyzer. The test results may also be synchronized to a remotely loaded application, such as an application on the user's mobile computing device. The software application may use the test results to evaluate a current fertility status of the user/subject, and may generate a fertility score. The systems and methods tracks the level changes of progesterone metabolites in a biological sample (e.g., urine) to confirm ovulation by calculating a gradient change in the progesterone metabolites over a consecutive period of days.

The devices and methods according to the present invention may quantitatively detect the concentration of progesterone metabolites, and track the level changes of progesterone metabolites during the menstruation cycle of women, which may assist in defining progesterone characteristics of women and checking whether the progesterone levels during the ovulation cycle are within a range, from low to high progesterone levels. Based on the quantitative results, the corpus luteum function and ovarian function can be evaluated. The causes of infertility like luteal insufficiency may also be evaluated and tracked. Advantageously, devices and methods according to the present invention enable a woman to track their cycle and treatment effectiveness in real time, without need of visiting a physician.

The devices and methods according to the present invention may also provide a user with information for tracking their pregnancy. During pregnancy, progesterone can be produced by the formed placenta, with levels of progesterone remaining elevated throughout pregnancy. These elevated levels prevent the body from producing additional eggs during pregnancy, and eventually promotes lactation. Devices and methods according to the present invention may track the progesterone metabolites changes during pregnancy, to assist in evaluating if there is a potential miscarriage or ectopic pregnancy. For women who have a high-risk pregnancy, or who are taking progesterone during pregnancy, this ability to track progesterone changes daily may prove helpful in early identification of potential issues.

The devices and methods according to the present invention may further be used to diagnose some progesterone-relevant diseases like adrenal disorder. Elevated 17-hydroxyprogesterone may be caused by congenital adrenal hyperplasia or ovarian or adrenal tumors. High levels of 17-OH progesterone can indicate a condition called congenital adrenal hyperplasia (CAH). The devices and methods according to the present invention may be configured to identify such elevated levels of these chemicals as a means for early warning of a potential disorder.

The devices and methods according to the present invention may further be used as a tool for remote real-time response to changes in hormone levels, and help doctors adjust medication; and may be used for testing any suitable biomarker based on the immunochromatographic assay with different antibodies.

Optionally, the fluorescent labels may be fluorescent molecules, fluorescent microspheres, or other fluorescent indicia that have similar absorption and emission wavelengths, with the fluorescent labels conjugated with antibodies by any suitable method. The antibodies may be anti-pregnanediol glucuronide, anti-pregnanediol, anti-progesterone, and other antibodies that can specifically recognize progesterone and progesterone metabolites or recognize the major (or all) progesterone metabolites. The conjugate pad may be provided as a single pad saturated with all the monoclonal anti-progesterone metabolites antibodies, or may be two or more separate pads, each saturated with different monoclonal anti-progesterone metabolites antibodies respectively. The testing zone may include one, two or more test line reactions with different anti-progesterone metabolites antibodies.

Both the foregoing general description and the following detailed description are exemplary and explanatory only and are intended to provide further explanation of the invention as claimed. The accompanying drawings are included to provide a further understanding of the invention; are incorporated in and constitute part of this specification; illustrate embodiments of the invention; and, together with the description, serve to explain the principles of the invention.

### BRIEF DESCRIPTION OF THE DRAWINGS

Further features and advantages of the invention can be ascertained from the following detailed description that is provided in connection with the drawings described below:

FIG. 1A depicts an overview of an immunoassay device in accordance with embodiments of the disclosure. FIG. 1B depicts an exploded top view of an immunoassay device in accordance with embodiments of the disclosure. FIG. 1C depicts an exploded bottom view of an immunoassay device in accordance with embodiments of the disclosure.

FIG. 2 demonstrates application of a fluid sample to an immunoassay device in accordance with embodiments of the disclosure.

FIGS. 3A-3C depict non-limiting examples of immunoassay devices in accordance with embodiments of the disclosure.

FIG. 4 depicts a non-limiting example of an immunoassay device in accordance with embodiments of the disclosure.

FIG. 5 depicts an exploded view of an exemplary diagnostic test system in accordance with embodiments of the disclosure.

FIG. 6 depicts a chamber for receiving and positioning an immunoassay device in accordance with embodiments of the disclosure.

FIG. 7 depicts a diagnostic test system in accordance with embodiments of the disclosure.

FIG. 8 depicts a diagnostic test system in operable communication with an immunoassay device in accordance with embodiments of the disclosure.

FIGS. 9A-B depict a non-limiting example of an optical configuration of a diagnostic test device in accordance with embodiments of the disclosure.

FIGS. 10A-B depict a detector module of a diagnostic test system in accordance with embodiments of the disclosure.

FIGS. 11A-C depict a diagnostic test system in accordance with embodiments of the disclosure.

FIG. 12A depicts a diagnostic test system in operable communication with a mobile device in accordance with embodiments of the disclosure. FIG. 12B depicts a non-limiting example of a mobile device displaying a mobile application in accordance with embodiments of the disclosure.

FIG. 13 depicts a method of using an immunoassay device in accordance with embodiments of the disclosure.

FIGS. 14A-D depict methods of using an immunoassay device in accordance with embodiments of the disclosure.

FIG. 15 depicts a non-limiting example of a labeling reaction performed on an immunoassay device in accordance with embodiments of the disclosure.

FIG. 16 depicts a non-limiting example of a competitive binding-based assay performed on an immunoassay device in accordance with embodiments of the disclosure.

FIG. 17 depicts a non-limiting example of a sandwich-based assay performed on an immunoassay device in accordance with embodiments of the disclosure.

FIGS. 18A-B depict non-limiting examples of a competitive binding-based assay performed on an immunoassay device in accordance with embodiments of the disclosure.

FIGS. 19A-B depict non-limiting examples of a sandwich-based assay performed on an immunoassay device in accordance with embodiments of the disclosure.

FIGS. 20A-B depict non-limiting examples of determining the presence of multiple analytes in a sample using an immunoassay device in accordance with embodiments of the disclosure.

FIGS. 21A-21B depict a non-limiting example of an immunoassay device in accordance with embodiments of the disclosure, showing: [A] a top plan view of the device; and [B] a bottom plan view of the device.

FIGS. 22A-22B depict a non-limiting example of a diagnostic test system in accordance with embodiments of the disclosure, showing: [A] a front perspective view of the system; and [B] a rear view of the system.

FIGS. 23A-23D depict a non-limiting example of a diagnostic test software application in accordance with embodiments of the disclosure, showing: [A] a home page screen; [B] a historical results screen; [C] a hormone curve screen; and [D] an edit cycle information screen.

### DETAILED DESCRIPTION OF THE INVENTION

The following disclosure discusses the present invention with reference to the examples shown in the accompanying drawings, though does not limit the invention to those examples. Reference is also made to US patent publication 2020/0300776, the entire contents of which is incorporated herein by reference.

The use of any and all examples, or exemplary language (e.g., “such as”) provided herein is intended merely to better illuminate the invention and does not pose a limitation on the scope of the invention unless otherwise claimed. No language in the specification should be construed as indicating any non-claimed element as essential or otherwise critical to the practice of the invention, unless otherwise made clear in context.

As used herein, the singular forms “a,” “an,” and “the” include plural referents unless the context clearly dictates otherwise. Unless indicated otherwise by context, the term “or” is to be understood as an inclusive “or.” Terms such as “first”, “second”, “third”, etc. when used to describe multiple devices or elements, are so used only to convey the relative actions, positioning and/or functions of the separate devices, and do not necessitate either a specific order for such devices or elements, or any specific quantity or ranking of such devices or elements.

The word “substantially”, as used herein with respect to any property or circumstance, refers to a degree of deviation that is sufficiently small so as to not appreciably detract from the identified property or circumstance. The exact degree of deviation allowable in a given circumstance will depend on the specific context, as would be understood by one having ordinary skill in the art.

Use of the terms “about” or “approximately” are intended to describe values above and/or below a stated value or range, as would be understood by one having ordinary skill in the art in the respective context. In some instances, this may encompass values in a range of approx. +/−10%; in other instances, there may be encompassed values in a range of approx. +/−5%; in yet other instances values in a range of approx. +/−2% may be encompassed; and in yet further instances, this may encompass values in a range of approx. +/−1%.

It will be understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof, unless indicated herein or otherwise clearly contradicted by context.

The term “analyte” as used herein may refer to any substance that is to be analyzed using the devise and methods provided herein. Non-limiting examples of analytes may include proteins, haptens, immunoglobulins, hormones, polynucleotides, steroids, drugs, infectious disease agents (e.g., of bacterial or viral origin), drugs of abuse, environmental agents, biological markers, and the like. In one case, the immunoassay detects at least a first analyte, wherein the first analyte is luteinizing hormone (LH). In another case, the immunoassay detects at least a first analyte, wherein the first analyte is human chorionic gonadotropin (hCG). In another case, the immunoassay detects at least a first analyte and a second analyte, wherein the first analyte is estrone-3-glucoronide (E3G) and the second analyte is luteinizing hormone (LH). In another case, the immunoassay detects at least a first analyte and a second analyte, wherein the first analyte is a surface antigen on a first viral particle (e.g., Influenza A) and the second analyte is a surface antigen on a second viral particle (e.g., Influenza B). In another case, the immunoassay detects at least a first analyte, wherein the first analyte is 25-hydroxyvitamin D, 25-hydroxyvitamin D2 [25(OH)D₂], or 25-hydroxyvitamin D3 [25(OH)D₃]. In another case, the immunoassay detects at least a first analyte and a second analyte, wherein the first analyte is triiodothyronine (T3) and the second analyte is thyroxine (T4). In another case, the immunoassay detects at least a first analyte, wherein the first analyte is an allergen. Non-limiting examples of allergens may include: Balsam of Peru, fruit, rice, garlic, oats, meat, milk, peanuts, fish, shellfish, soy, tree nuts, wheat, hot peppers, gluten, eggs, tartrazine, sulfites, tetracycline, phenytoin, carbamazepine, penicillin, cephalosporins, sulfonamides, non-steroidal anti-inflammatories (e.g., cromolyn sodium, nedocromil sodium, etc.), intravenous contrast dye, local anesthetics, pollen, cat allergens, dog allergens, insect stings, mold, perfume, cosmetics, semen, latex, water, house dust mites, nickel, gold, chromium, cobalt chloride, formaldehyde, photographic developers, fungicide, dimethylaminopropylamine, paraphenylenediamine, glyceryl monothioglycolate, toluenesulfonomide formaldehyde.

The terms “individual”, “host”, “subject”, and “patient”, as may be used interchangeably herein, refer to a plant, fungi, eubacteria, archaebacteria, protist, or animal. The animal can be a fish (e.g., a zebrafish), a mammal, including though not limited to a dog, cat, horse, cow, mouse, rat, pig or a primate. Primates may include, for example, human beings, as well as chimpanzee, orangutan, gorilla, rodents, such as mice and rats, and other laboratory animals. The animal may be male or female, and may be of any age. The female may be pregnant, suspected of being pregnant, or planning to become pregnant. The female may be ovulating. The subject may be an organism, either a single-celled or multi-cellular organism. The subject may be cultured cells, which may be primary cells or cells from an established cell line, among others. Examples of cell lines include, but are not limited to, 293-T human kidney cells, A2870 human ovary cells, A431 human epithelium, B35 rat neuroblastoma cells, BHK-21 hamster kidney cells, BR293 human breast cells, CHO Chinese hamster ovary cells, CORL23 human lung cells, HeLa cells, or Jurkat cells.

Recitations of value ranges herein, unless indicated otherwise, serve as shorthand for referring individually to each separate value falling within the respective ranges, including the endpoints of the range, each separate value within the range, and all intermediate ranges subsumed by the overall range, with each incorporated into the specification as if individually recited herein.

Unless indicated otherwise, or clearly contradicted by context, methods described herein can be performed with the individual steps executed in any suitable order, including: the precise order disclosed, without any intermediate steps or with one or more further steps interposed between the disclosed steps; with the disclosed steps performed in an order other than the exact order disclosed; with one or more steps performed simultaneously; and with one or more disclosed steps omitted.

Provided herein are lateral flow immunoassay devices (e.g., test wands), diagnostic reader devices (e.g., an analyzer device) configured to receive an immunoassay device and to provide an output based on the results of the immunoassay, and kits comprising immunoassay devices and/or diagnostic reader devices. Methods of using the immunoassay devices and diagnostic reader devices are also disclosed. The disclosed devices, kits and methods may be used to perform immunoassay tests on a biological sample, for example, to diagnose a disease or to provide information regarding a biological state or condition of a subject (e.g., fertility status).

The immunoassay devices use immunochromatographic assays with new fluorescent labeling technology, and the diagnostic reader devices are configured to specifically identify signals from the immunoassay devices wands, and sync information to mobile devices. Application software is also provided for executing processes to achieve these ends. Systems according to the present invention may quantitatively detect and track changes in a level of progesterone metabolites in urine, which may be used to confirm ovulation or evaluate menstruation status of a subject.

In one aspect, the present invention is inclusive of testing wands (e.g., lateral flow immunoassay devices) and analyzer devices (diagnostic reader devices) configured to detect progesterone metabolite in urine for use in tracking and confirming ovulation, evaluating ovulatory function of the ovaries, assessing corpus luteum sustainability, and assisting in defining fertile and non-fertile days in the menstrual cycle of a user. Optionally, the testing wands and analyzer device may track other hormones and hormone metabolites. Unlike conventional systems, which rely on a predetermined and fixed threshold, testing systems according to the present invention quantitatively establish a dynamic threshold personalized to a specific user, which enables a more personalized and accurate tracking of hormonal changes throughout the specific user's menstrual cycle.

Testing systems according to the present invention include an analyzer and a number of test wands that employ an immunochromatography assay with fluorescent labeling. The test wands including a test strip defining a fluid flow path for receiving a biological sample, conveying the biological sample through an immunoassay in a step-by-step process, and outputting one or more signals for communication of an immunoassay result. The analyzer is configured to identify signals from the test wands, analyze immunoassay results from the test wands, and sync information to one or more remote devices, such as a user's mobile device and/or personal computing device.

The analyzer is a palm-sized portable device configured to assess immunoassay tests based on fluorescence scanning technology. The analyzer has a wand insertion port for insertion of a test wand, a chip reading device for identifying test wand information, a scanning device to scan for a test signal of a reaction on a membrane of wand, a display screen for displaying test results, and communications systems (e.g., Bluetooth synchronizing) for communicating with one or more remote devices. The analyzer is pre-programmed to recognize and identify different wands, each of which may be adapted for the detection of different analytes. The test wands may be provided with an immunoassay configured to detect the presence of anywhere from 1 to 10 or more analytes in a sample. The analyzer may be programmed to communicate with a number of remote devices, including a mobile device, such that the remote devices may record and track immunoassay results.

Each test wand includes a test strip that provides a flow path for conveyance of a fluid biological sample that is to be subject to an immunoassay. Generally, the test strip is composed of a material suitable for performing the one or more immunoassays, for use as a substrate for immobilizing or otherwise providing various reagents to perform the immunoassay, and for a lateral flow of a fluid biological sample across the test strip. In some examples, the test strip may be composed of a material comprising a plurality of capillary beds such that, when contacted with a fluid, the fluid is transported laterally across the test strip. Non-limiting examples of materials suitable for use in forming a test strip include porous paper, a membrane polymer such as nitrocellulose, polyvinylidene fluoride, nylon, Fusion 5™, or polyethersulfone. The test strip may comprise a plurality of discrete regions, each positioned laterally along the flow path, with each region having a variety of reagents for performing a step in an immunoassay such that as a fluid biological sample flows along the test strip from, from a first, proximal end to a second, distal end, an immunoassay is conducted on the biological sample in a step-by-step fashion.

The flow path comprises a sample zone (e.g., a sample pad) for receiving a biological sample; a labeling zone (e.g., a conjugate pad) provided with one or more detection reagents (e.g., anti-progesterone metabolites and/or other antibodies conjugated to a fluorescent label); a capture zone (e.g., a testing zone) provided with one or more capture reagents; a control zone provided with one or more control reagents; and an absorbent paper or other wicking material to facilitate chromatography. The capture zone and control zone may be provided on a common membrane.

The sample zone is a region of the flow path configured to receive or accept a biological sample. The sample zone is located at or near a first, proximal end of the flow path, which in the illustrated example coincides with a first, proximal end of the test strip and the test wand as a whole. The sample zone may be adapted to receive a biological sample in the form of a fluid, for example, by inserting the sample zone into a container holding the fluid sample, pipetting the fluid sample directly onto the sample zone, or holding the sample zone under a flow stream of the fluid sample (e.g., a urine stream).

In some examples, the sample zone may include a pad or other contact surface. A pad may be composed of a woven mesh or a fibrous material such as a cellulose filter, polyesters, or glass fiber. The sample zone may further include, without limitation, pH and ionic strength modifiers such as buffer salts (e.g., Tris), viscosity enhancers to modulate flow properties, blocking and resolubilization agents (e.g., proteins, such as albumin; detergents; surfactants, such as Triton X-100, Tween-20; and/or filtering agents, such as those to filter whole blood.

The labeling zone is positioned on the flow path of the test strip, downstream of the sample zone such that a fluid biological sample introduced to the sample zone flows downstream from the sample zone to the labeling zone. The labeling zone may be provided in the form of a conjugate pad that is composed of, for example, glass fibers, cellulose filters, or surface-modified polyester. The labeling zone may comprise one or more detection reagents. The detection reagents may be adsorbed on a surface of the labeling zone, and are freely mobile or mobilizable when in a wet state, for example, when a liquid saturates the labeling zone.

The one or more detection reagents may be chosen for binding to specific analytes that are targeted for detection in a biological sample, so as to form an analyte-reagent complex. Generally, the one or more detection reagents will have specific binding activity for the respectively targeted analytes. For example, in a case where there are first and second detection reagents, the first detection reagent may specifically or selectively bind to a first analyte, but not a second analyte, while the second detection reagent may specifically or selectively bind to the second analyte, but not the first analyte. Any number of detection reagents may be used to detect a desirable number of analytes, with each detection reagent having a specific binding activity for a respective analyte.

A detection reagent may be conjugated or otherwise attached to a detectable label that generates a signal (e.g., such as an optical signal from a fluorescent label). For example, a first detection reagent may be conjugated to a first detectable label and a second detection reagent may be conjugated to a second detectable label. A detectable label may be a fluorophore, an enzyme, a quencher, an enzyme inhibitor, a radioactive label, one member of a binding pair or any combination thereof. In some cases, the detectable label is composed of fluorescent molecules, e.g., fluorophores. Non-limiting examples of fluorophores suitable for use with the test wand may include: fluorescein (FITC) and fluorescein derivatives such as FAM, VIC, and JOE, 5-(2′-aminoethyl)aminonaphthalene-1-sulphonic acid (EDANS), coumarin and coumarin derivatives, Lucifer yellow, NED, Texas red, tetramethylrhodamine, tetrachloro-6-carboxyfluoroscein, 5 carboxyrhodamine, cyanine dye, Alexa Fluor 350, Alexa Fluor 647, Oregon Green, Alexa Fluor 405, Alexa Fluor 680, Alexa Fluor 488, Alexa Fluor 750, Cy3, Alexa Fluor 532, Pacific Blue, Pacific Orange, Alexa Fluor 546, Tetramethylrhodamine (TRITC), Alexa Fluor 555, BODIPY FL, Texas Red, Alexa Fluor 568, Pacific Green, Cy5, Alexa Fluor 594, Super Bright 436, Super Bright 600, Super Bright 645, Super Bright 702, DAPI, SYTOX Green, SYTO 9, TO-PRO-3, Propidium Iodide, Qdot 525, Qdot 565, Qdot 605, Qdot 655, Qdot 705, Qdot 800, R-Phycoerythrin (R-PE), Allophycocyanin (APC), cyan fluorescent protein (CFP) and derivatives thereof, green fluorescent protein (GFP) and derivatives thereof, red fluorescent protein (RFP) and derivatives thereof, and the like. Any fluorophore with an excitation wavelength of between about 300 nm and about 900 nm is envisioned herein.

In some cases, there may be a single detectable label, and in other cases there may be separate first and second detectable labels. A detection reagent may be present at a labeling zone in an amount that is in excess of the amount of analyte present in the biological sample. For example, a detection reagent may be present at the labeling zone at an amount anywhere from 2× to 1000×, or greater, than the amount of the respective analyte present in the biological sample.

The labeling zone may further comprise additional chemicals, including, but not limited to, bovine serum albumin (BSA), polyvinylpyrrolidine 40 (PVP40), Triton X-100, sucrose, Tween-20, Tris, fetal bovine serum (FBS), or any combination thereof.

The capture zone is positioned on the flow path of the test strip, downstream of the labeling zone such that a fluid biological sample flows downstream from the labeling zone to the capture zone. The capture zone may comprise one or more discrete capture regions. Generally, the number of capture regions employed will depend on the number of analytes to be detected. For example, if two analytes are to be detected, the device will have two capture regions, if three analytes are to be detected, the device will have three capture regions, and so on.

A capture region will have a capture reagent immobilized thereon. An immobilized capture reagent is made to bind with and immobilize a target reagent or complex. In this way, an otherwise mobile target reagent or complex that flows to the capture region from the labeling region and binds with a corresponding capture reagent will become immobilized at the capture region.

In some cases, a capture region may be configured to perform a competitive binding-like assay as described herein. In a particular example, a capture reagent may specifically bind to a non-complexed detection reagent (i.e., when a detection reagent has not formed a complex with a respective analyte at the labeling zone). In such cases, the capture reagent does not bind to a detection reagent that is bound to an analyte (i.e., such a capture reagent does not bind to an analyte-reagent complex, when formed). A capture reagent may not displace an analyte from a detection reagent when the analyte is bound to the detection reagent. Thus, the amount of an analyte present in a biological sample may determine an amount of a corresponding detection reagent that is capable of binding to the respective reagent at the capture region. For example, when an amount of an analyte present in a biological sample is low, or when the analyte is absent from the biological sample, the detection reagent may be mostly in a non-complexed state when it reaches the capture region, and therefore more non-complexed detection reagent may be available for binding to the capture reagent. On the other hand, when the amount of analyte present in the biological sample is high, the detection reagent may be mostly present at the capture region in a complex with the analyte, and therefore, less detection reagent may be available for binding to the capture reagent.

In some examples, the capture region may be configured to perform a sandwich binding assay. In some cases, the capture reagent may specifically bind to the analyte-reagent complex (i.e., when a detection reagent has formed a complex with a respective analyte at the labeling zone). In such cases, the capture reagent does not bind to the analyte or the detection reagent, unless they are present in a complex with one another (i.e., such a capture reagent binds to an analyte-reagent complex, when formed). Thus, the amount of analyte present in the biological sample may determine the amount of analyte-reagent complex that is capable of binding to the capture reagent at the capture region. For example, when an amount of analyte present in the biological sample is low or is absent in the biological sample, the detection reagent may be mostly in a non-complexed state when it reaches the capture region, with less analyte-reagent complex present, such that less analyte-reagent complex is available for binding to the capture reagent at the capture region. When an amount of analyte present in the biological sample is high, the detection reagent may be mostly present at the capture region in a complex with the analyte, and therefore, more analyte-reagent complex may be available for binding to the capture reagent at the capture region.

An optical signal from the detectable label conjugated to the detection reagent (e.g., a fluorescent signal from a fluorescent label) is detectable at the capture region. For example, a test wand with the test strip having a capture region may be insertable into an analyzer device as described herein for detection of an optical signal at the capture region. The optical signal may be a readout for the amount of an analyte present in the sample. In one example, the optical signal may inform an amount of detection reagent bound to a capture reagent. In such cases, the optical signal may increase when an amount of analyte present in the biological sample is low, with the optical signal decreasing when an amount of the analyte present in the biological sample is high. In another example, the optical signal may inform an amount of analyte-reagent complex bound to a capture reagent. In such cases, the optical signal may increase when an amount of analyte present in the biological sample is high, with the optical signal decreasing when an amount of the analyte present in the biological sample is low. The optical signal at the capture region may be proportional to the amount of analyte present in the biological sample (e.g., directly proportional, inversely proportional, exponentially proportional, logarithmically proportional, etc.).

In some instances, a first capture region and a second capture region may be present on a flow path of a test strip. In one example, the first capture region may be upstream of the second capture region. In another example, the first capture region may be downstream of the second capture region. When the device utilizes two capture regions, the order of the capture regions will be unimportant, so long as one capture region utilizes a sandwich binding assay and the other utilizes a competitive binding assay as described above.

The capture zone may comprise any number of capture regions, with capture reagents immobilized on the respective capture regions, with the individual capture reagents having a binding operation (e.g., binding with complexed or non-complexed detection reagents), and with each capture region generating an optical signal based on a binding state of the capture reagent as informing the presence of an analyte, as described above.

The control zone is positioned on the flow path of the test strip, downstream of the capture zone such that a fluid biological sample flows downstream from the capture zone to the control zone. The control zone may comprise one or more control regions, each with a control reagent immobilized thereon. The control reagent is provided for binding to a detection reagent, regardless if the detection reagent is in a complexed or non-complexed state, with the specific control reagent selected based on the identity of the detection reagent. Essentially any ligand with binding affinity for the detection reagent may be utilized. The control region may be configured to bind with excess detection reagent that did not bind to capture reagent in the capture zone.

An optical signal from the detectable label conjugated to the detection reagent (e.g., a fluorescent signal from a fluorescent label) is detectable at the control region, just as with the capture region. In some examples, when there is a high amount of analyte in a biological sample, and when the capture region is provided with capture reagents that bind with detection reagents in a non-complexed state only, then a high amount of detection reagents will pass through the capture region in a complexed state and be available for binding with the control reagents in the control region. In some examples, when there is a high amount of analyte in a biological sample, and when the capture region is provided with capture reagents that bind with detection reagents in a complexed state only, then a low amount of detection reagents (if any) will pass through the capture region in a non-complexed state and be available for binding with the control reagents in the control region. In some examples, when there is a low amount of analyte in a biological sample, and when the capture region is provided with capture reagents that bind with detection reagents in a non-complexed state only, then a low amount of detection reagents will pass through the capture region in a complexed state and be available for binding with the control reagents in the control region. In some examples, when there is a low amount of analyte in a biological sample, and when the capture region is provided with capture reagents that bind with detection reagents in a complexed state only, then a high amount of detection reagents (if any) will pass through the capture region in a non-complexed state and be available for binding with the control reagents in the control region.

In some examples, in cases where a biological sample is assessed for the presence of two or more analytes, using two or more detection reagents corresponding to the respective analytes, the control reagent may be adapted to bind with each of the two or more detection reagents such that the control region may immobilize each of the respective detection reagents, be they in complexed or non-complexed states.

In some examples, the control zone may comprise two or more control regions positioned sequentially along the flow path such that a biological sample flows through the several control regions. Each successive control region will have a control reagent immobilized thereon that binds to each of the detection reagents. In some cases, the subsequent control regions may have the same capture reagent as the first control region, and in other cases each subsequent control region may have a different capture reagent from each prior control region. In cases where two or more control region are used, each subsequent control region will capture any excess detection reagents that were not immobilized in the prior control region(s). As the biological sample flows through successive control regions, the detection reagent(s) will be increasingly depleted from the biological sample such that the signal generated and detectable at each successive control region should continually decrease from one control region to the next.

The amount of a control reagent may be the same, or may vary from one control region to the next. When made to differ, the amount of control reagent may either increase or decreasing between successive control regions, with the increasing/decreasing difference being anywhere from 1.5× to or greater between the successive control regions. The amounts of control reagent in the one or more control regions may be customized or tailored to increase the sensitivity or precision of the immunoassay as needed.

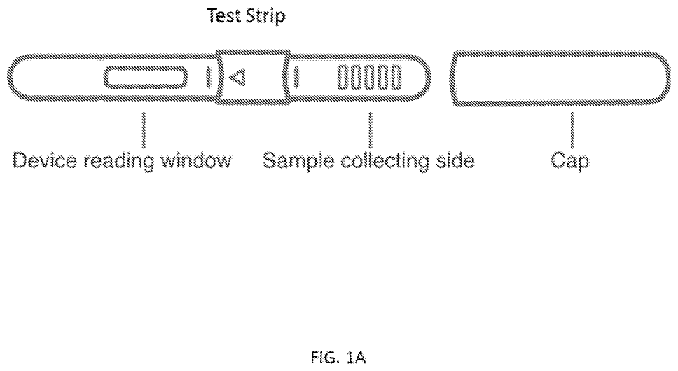

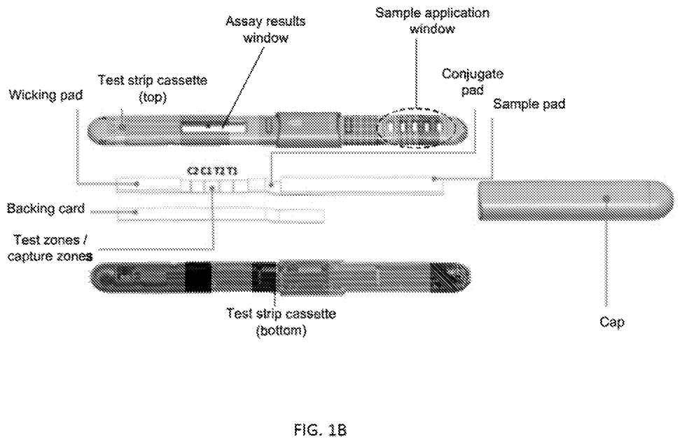

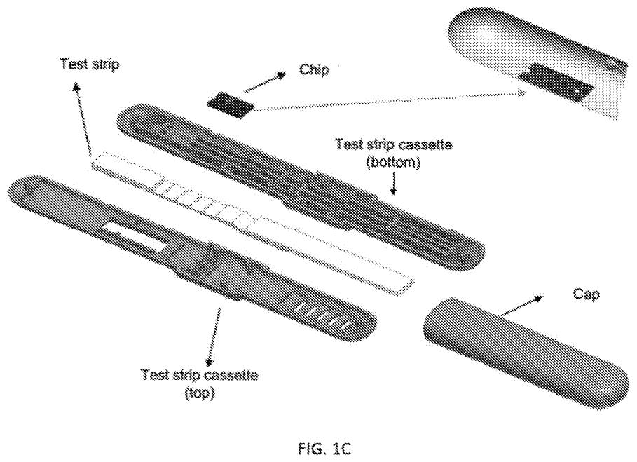

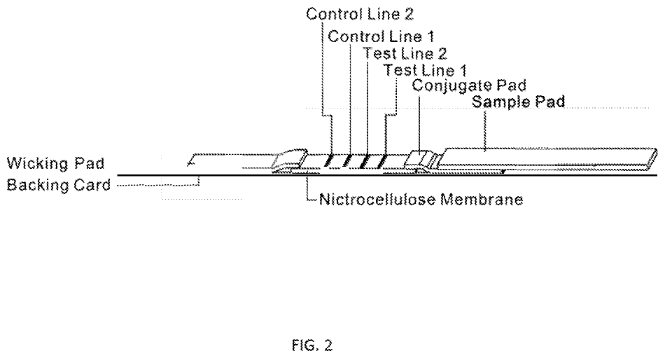

The testing wand may further include one or more additional features, as depicted in FIGS. 1B and 1C. The test wand may further include a wicking pad, generally located at the distal end of the test wand, downstream from the test strip. The wicking pad may be composed of, e.g., filter paper.

As also depicted in FIGS. 1B and 1C, the test wand may also include a test strip cassette for supporting and/or protecting the test strip. The cassette may be composed of a sturdy material such as plastic (e.g., high-impact polystyrene). The cassette may prevent a user from applying the sample anywhere other than the sample pad, may protect the device from inadvertent splashing of a sample onto the test strip (e.g., when the device is applied to a urine stream), and may protect the sensitive areas of the test strip (e.g., the capture and control zones). The cassette may include various openings or windows along the device. For example, the cassette may include, at a proximal end, a sample application window for applying a fluid biological sample to the sample pad. The cassette may further include an assay results window for visualization of the assay results. The assay results window may be positioned on the device directly above the capture zone and/or control zone such that a detectable signal can be visualized or read (e.g., by a diagnostic test system). The cassette may be of a certain size and shape so as to be compatible with an analyzer device of the disclosure (or another diagnostic test device), such that the cassette may be inserted into a port or receiver of the analyzer device. The test wand may further include a cap composed of, e.g., polypropylene, designed to be inserted over the proximal end of the device to cover the sample application window.

The test wand may further include a readable chip. The readable chip may be positioned on the underside of the device, for example, embedded within or otherwise attached to the bottom of the test strip cassette. The readable chip may be configured to be in electronic communication with a diagnostic test system, after the device is inserted into the diagnostic test system. The readable chip may be recognized by the diagnostic test system such that upon insertion of the test wand into the diagnostic test system, the diagnostic test system automatically performs one or more defined operations.

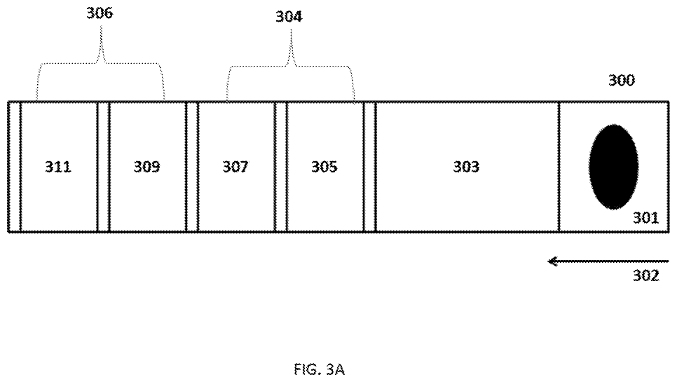

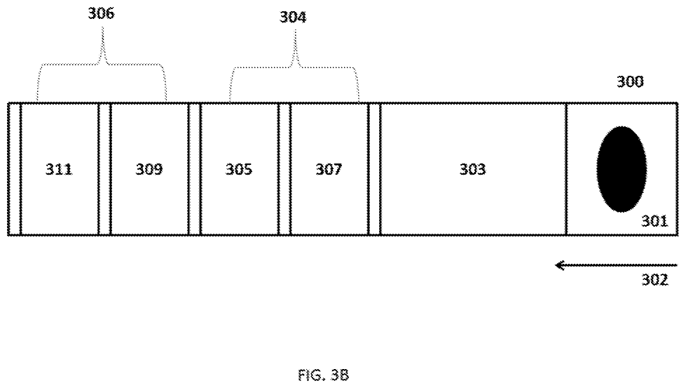

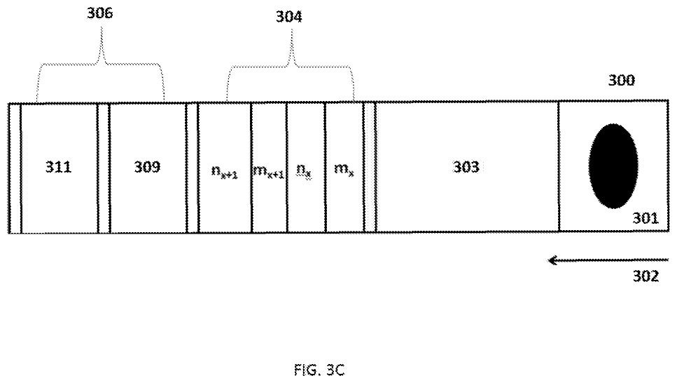

FIGS. 3A & B depict non-limiting examples of a test strip 300 in accordance with various examples as described herein. The test strip 300 may define a flow path comprising various zones and/or regions for conducting an immunoassay, in accordance with the present disclosure. The various zones and/or regions may be positioned along the flow path of the test strip such that a fluid biological sample may be flowed (e.g., by capillarity) from a first end of the test strip to a second end of the test strip, as depicted by directional arrow 302. The test wand may include a sample zone 301 positioned at a first end of the test strip, and which may be configured to receive a biological sample as described herein. The test wand may further comprise a labeling zone 303 (e.g., a conjugate pad) positioned downstream of the sample zone on the flow path of the test strip. The labeling zone 303 may contain one or more detection reagents for labeling one or more analytes when present in the sample. The test wand may further comprise a capture zone 304 downstream on the flow path from the labeling zone 303. The capture zone may further comprise one or more capture regions, for example, a first capture region 305 and a second capture region 307. Each of the one or more capture regions may contain a capture reagent for performing an immunoassay. The immunoassay may be a sandwich-based immunoassay or a competitive binding-based immunoassay. In some cases, the first capture region 305 contains reagents suitable to perform a competitive binding-based immunoassay and the second capture region 307 contains reagents suitable to perform a sandwich-based immunoassay. In other cases, the first capture region 305 contains reagents suitable to perform a sandwich-based immunoassay and the second capture region 307 contains reagents suitable to perform a competitive binding-based immunoassay. As depicted in FIG. 3A, the first capture region 305 may be positioned upstream of the second capture region 307 on the flow path of the test strip. As depicted in FIG. 3B, the first capture region 305 may be positioned downstream of the second capture region 307 on the flow path of the test strip. The test wand may further comprise a control zone 306 positioned downstream from the capture zone 304 on the flow path of the test strip. The control zone may further comprise one or more control regions, for example, a first control region 309 and a second control region 311. The second control region 311 may be positioned downstream of the first control region 309 on the flow path of the test strip. With reference to FIG. 3C, the test strip may contain a plurality of first capture regions (m) and a plurality of second capture regions (n).

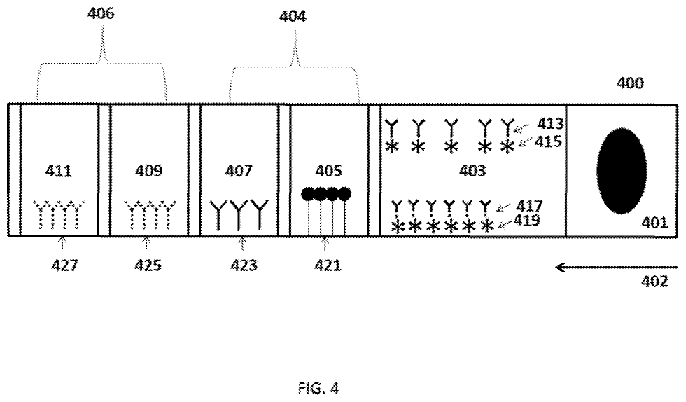

FIG. 4 depicts a non-limiting example of immunoassay reagents provided on a test strip 400 in accordance with the disclosure. The test strip in this example comprises a sample zone 401 positioned at a first end, with a labeling zone 403 positioned downstream of the sample zone 401 on a flow path 402. The labeling zone 403 may comprise a mobilizable first detection reagent 413 conjugated to a first detectable label 415. The first detection reagent may be capable of specifically binding to a first analyte when present in the sample. The labeling zone 403 may further comprise a mobilizable second detection reagent 417 conjugated to a second detectable label 419. The second detection reagent may be capable of specifically binding to a second analyte when present in the sample. The test strip 400 further comprises a capture zone 404 comprising a first capture region 405 and a second capture region 407. The first capture region 405 has, immobilized thereon, a first capture reagent 421, which may be, e.g., an antigen. The second capture region 407 has, immobilized thereon, a second capture reagent 423, which may be, e.g., an antibody or antibody fragment. The test strip 400 further comprises a control zone 406 comprising a first control region 409 and a second control region 411. The first control region 409 has, immobilized thereon, a first control reagent 425, which may be, e.g., an antibody or antibody fragment. The second control region 411 has, immobilized thereon, a second control reagent 427, which may be, e.g., an antibody or antibody fragment.

The analyzer device comprises: a housing, comprising: a) a port for receiving an assay device (e.g., a test wand as disclosed herein), said assay device comprising two or more capture regions; b) a reader comprising: i) one or more light sources for illuminating said two or more capture regions; ii) one or more light detectors for detecting optical signals from said two or more capture regions; and c) a data analyzer having one or more processors configured to: A) receive said optical signals; and B) determine an amount of at least a first analyte and a second analyte present in a biological sample based on said optical signals, wherein an optical signal of a first of said two or more capture regions increases with decreasing amounts of said first analyte present in said biological sample, and an optical signal of a second of said two or more capture regions increases with increasing amounts of said second analyte present in said biological sample.

The diagnostic test system may include a housing for containing the components of the system. The housing can be constructed of any suitable material. The housing may be configured to receive an immunoassay device of the disclosure. For example, the housing may include a port or opening for receiving the immunoassay device. The system may further include, contained within the housing, a reader device. The reader device may include one or more light sources for illuminating the immunoassay device or a region of the immunoassay device. In one non-limiting example, the one or more light sources are configured to illuminate the capture zone of an immunoassay device of the disclosure. The type of light source suitable for use with the immunoassay devices will depend on the chemistry of the immunoassay device. In one particular example, the one or more light sources are used to illuminate a detectable label provided by the immunoassay device. In a particular example, the detectable label provided on the immunoassay device is a fluorophore, and therefore, the one or more light sources of the reader device should include a fluorescent light source (e.g., a light-emitting diode (LED)). It is to be understood that the wavelength of light provided by the light source of the reader device should be selected based on the excitation wavelength of the detectable label, and can readily be selected by a person of skill in the art.

The reader may be configured to illuminate the capture zone and/or the control zone of an immunoassay device of the disclosure. For example, the reader may be configured to illuminate the first capture region, the second capture region, the first control region, the second control region, or any combination thereof. In some cases, the reader is configured to scan across the test strip of an immunoassay device. In such cases where the immunoassay device utilizes a single fluorophore, the reader may contain a single fluorescent light source. In cases where the immunoassay device utilizes more than one fluorophore, the reader may contain more than one fluorescent light source.

The reader may further comprise one or more light detectors (e.g., a photodetector) for detecting optical signals from the immunoassay device. Generally speaking, the one or more light detectors should be capable of distinguishing between emitted light at a first discrete position and a second discrete position on the immunoassay device. This may be accomplished by, e.g., the one or more light sources scanning across the test strip of the immunoassay device and determining the position of the emitted light on the immunoassay device.

The diagnostic test device may further comprise a data analyzer. The data analyzer may have one or more processors configured to receive an optical signal. In some cases, the data analyzer is in operable communication with a reader device. The data analyzer may be configured to determine an amount of analytes present in a sample, for example, by measuring an amount of optical signal produced at the capture zone of an immunoassay device. For example, the data analyzer may be configured to calculate the area under the curve of a signal intensity plot. The data analyzer may further be configured to determine the differences between signal intensities among the multiple discrete regions on the test strip. For example, the data analyzer may be configured to determine the difference between the signal intensity at the first capture region and the signal intensity at the second control region. The data analyzer may further be configured to determine the difference between the signal intensity at the second capture region and the signal intensity at the first control region. The data analyzer may further be configured to calculate an amount or concentration of the analytes present in the sample. The data analyzer may be further configured to detect a binary optical pattern.

The binary optical pattern can be generated by two fluorescent materials which excitation and/or emission spectrum differs in wavelength. In some cases, the binary optical pattern can be generated by one fluorescent material and one light absorbent material. The detection reagents may be conjugated with the two types of materials respectively and can be captured in the same capture zone, such that the capture zone may generate two different optical signal patterns in the data analyzer.

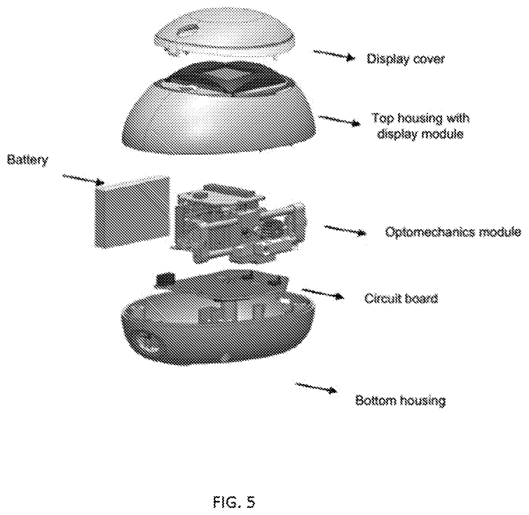

FIG. 5 depicts an exploded view of an exemplary diagnostic test system of the disclosure. The system may comprise a housing for containing the electronic components of the system. The system may have a top housing and a bottom housing. The top housing may comprise a display module for displaying the results of an immunoassay as described herein. The system may further comprise a display cover. The system may further comprise a battery. The system may further comprise an optomechanics module. The optomechanics module may comprise the one or more light sources and one or more light detectors as described above. The system may further comprise a circuit board containing electronic components.

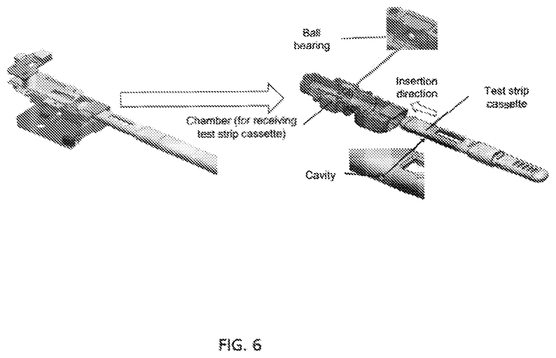

FIG. 6 depicts a non-limiting example of a receiving port or chamber of a diagnostic test system for receiving an immunoassay test device of the disclosure. The cassette or housing of the immunoassay test device may include a cavity. The chamber or receiving port of the diagnostic test system may include a ball bearing contained within the inner wall of the chamber. The ball bearing may hook or latch into the cavity of the test device, thereby locking the immunoassay test device into the receiving chamber of the diagnostic test system.

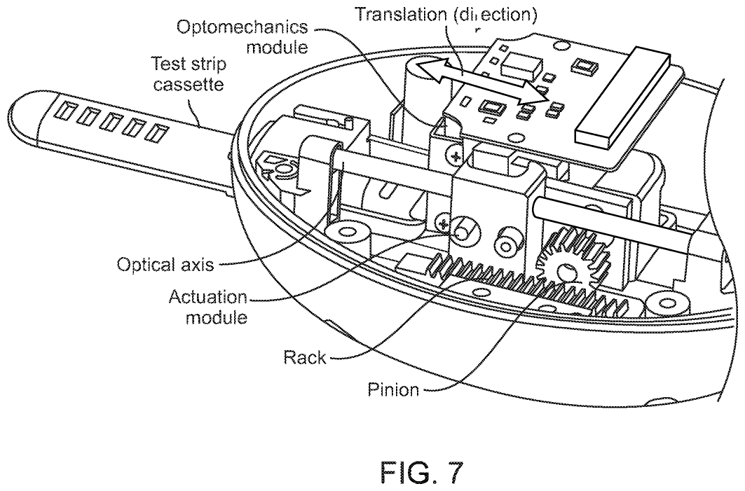

FIG. 7 depicts inner components of a diagnostic test system, in accordance with embodiments of the disclosure. The diagnostic test system may include an optomechanics module comprising the one or more light sources for illuminating the test strip of the immunoassay device. The optomechanics module may be movable across an optical axis such that the optomechanics module moves laterally across the test strip of the immunoassay device, thereby scanning the test strip. The diagnostic test system may further comprise an actuation module. The actuation module may comprise one or more motors configured to actuate/move the optomechanics module. In some embodiments, the motors may be coupled to a rack and pinion mechanism that is configured to translate the optomechanics module along one or more directions. For example, the optomechanics module can be translated along a longitudinal axis of the test strip of the immunoassay device. The direction(s) of translation may or may not be orthogonal to an optical axis of the optomechanics module. The direction(s) of translation may be parallel to the longitudinal axis of the test strip, and the optical axis may be orthogonal to the longitudinal axis or a planar surface of the test strip. In some cases, the direction(s) of translation need not be parallel to the longitudinal axis of the test strip, and the optical axis need not be orthogonal to the longitudinal axis (or a planar surface) of the test strip. For example, the direction(s) of translation and/or the optical axis may be at an oblique angle relative to the longitudinal axis of the test strip.

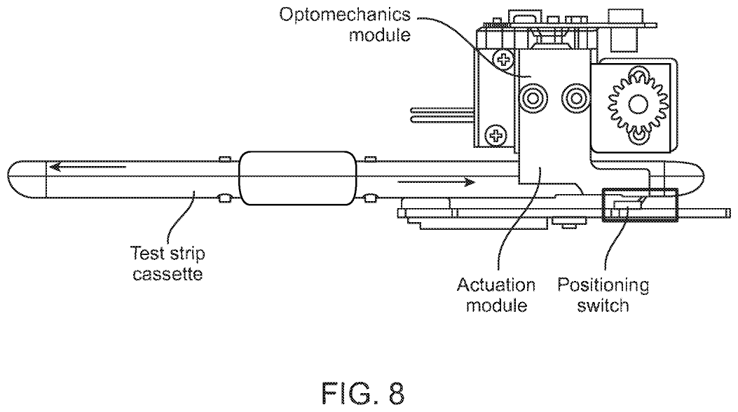

FIG. 8 depicts an immunoassay device inserted into the receiving chamber of a diagnostic test system of the disclosure. The diagnostic test system may include a positioning switch. The positioning switch may be configured to switch between an open position and a closed position. The positioning switch may be in the open position when the immunoassay device is not inserted into the receiving chamber. The position switch may be in the closed position when the immunoassay device is inserted into the receiving chamber and located at a predetermined position within the receiving chamber. The immunoassay device may be located at the predetermined position, for example when a cavity on the test strip cassette engages a ball-bearing within the chamber (see FIG. 6). The positioning switch may be configured to activate the actuation module and the optomechanics module to scan the test strip on the immunoassay device when the positioning switch is in the closed position. In some cases, the scan of the test strip may be terminated if the scan has been completed, or when the positioning switch has been moved to the open position.

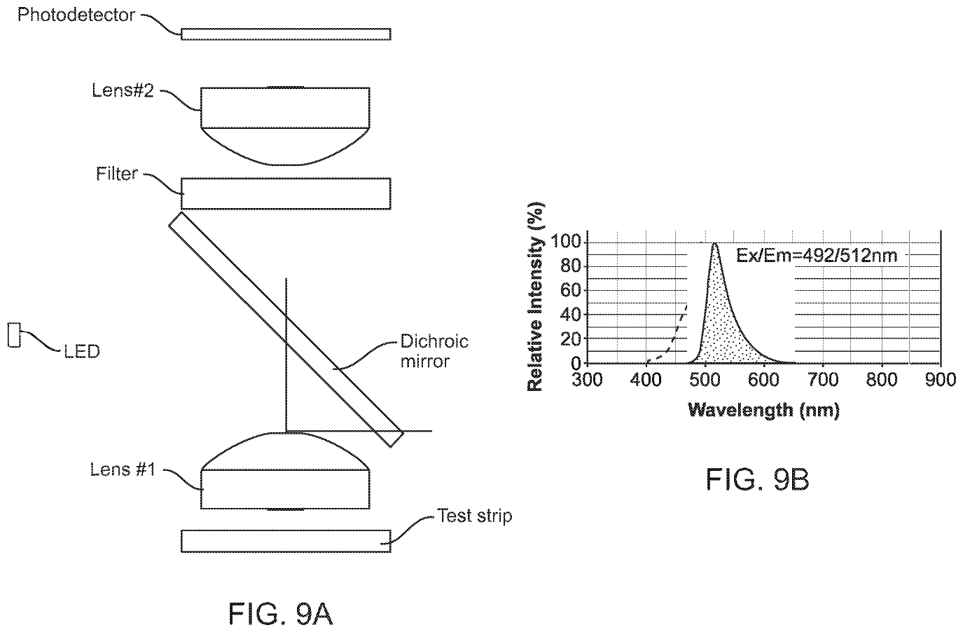

FIG. 9A depicts a non-limiting example of an optical configuration suitable for use with the diagnostic test system and positioning of the optics above a test strip of an immunoassay device. The optical configuration may include a light source (e.g., a light-emitting diode (LED) for illuminating the test strip. The optical configuration may further include one or more lens, filters, optical beamsplitters, or any combination thereof. The optical configuration may further include a photodetector for detecting an optical signal from the immunoassay device. FIG. 9B depicts an example of an excitation/emission spectra with an excitation wavelength of 492 nm and an emission wavelength of 512 nm.

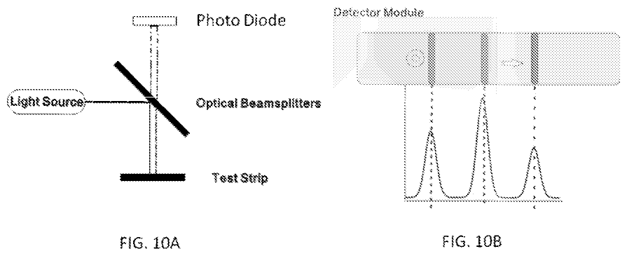

FIG. 10A depicts positioning of optical configuration at a position (e.g., Position A) of an immunoassay device. As a diagnostic test system of the disclosure moves longitudinally and scans across the test strip of the immunoassay device, an emission spectrum is generated corresponding to increased optical signal at various regions along the test strip, as shown in FIG. 10B.

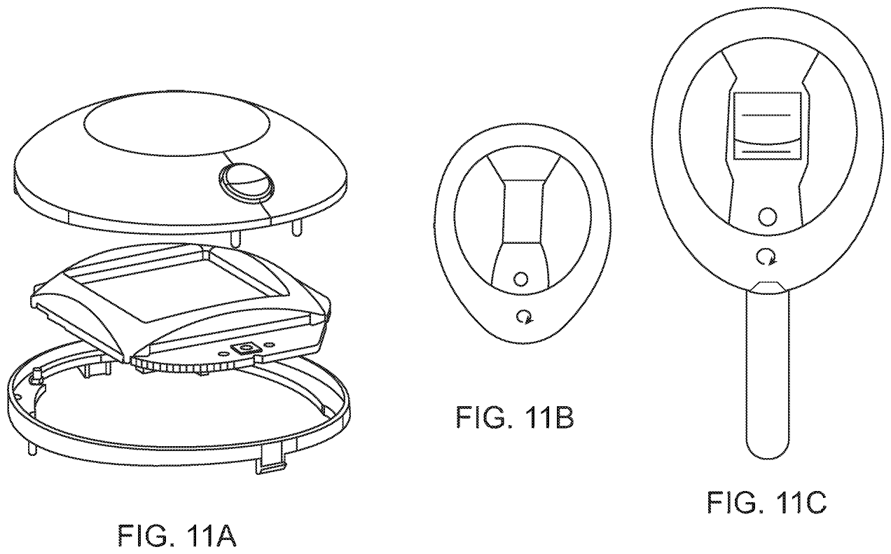

FIGS. 11A and B depict non-limiting examples of a diagnostic test system, demonstrating various design elements of the device. FIG. 11C depicts a non-limiting example of a top view of a diagnostic test system with an immunoassay test device inserted into the receiving chamber of the system.

In some cases, the diagnostic test device generates measurement results (e.g., concentration or relative amounts of analytes present in the sample) from a completed assay performed on the test device, as described throughout. In some cases, the diagnostic test device displays the measurement results on the device screen. Data containing the measurement results can be transmitted from the diagnostic test device to a mobile device and/or to a server. The data may be transmitted via one or more wireless or wired communication channels. The wireless communication channels may comprise Bluetooth®, WiFi, 3G, 4G, and/or 5G networks.

The data containing the measurement results may be stored in a memory on the diagnostic test device when the diagnostic test device is not in operable communication with the mobile device and/or the server. The data may be transmitted from the diagnostic test device to the mobile device and/or the server when operable communication between the diagnostic test device and the mobile device and/or the server is re-established.

A network can be configured to provide communication between the various components of the embodiments described herein. The network may be implemented, in some embodiments, as one or more networks that connect devices and/or components in the network layout for allowing communication between them. For example, one or more diagnostic test devices, mobile devices and/or servers may be in operable communication with one another over a network. Direct communications may be provided between two or more of the above components. The direct communications may occur without requiring any intermediary device or network. Indirect communications may be provided between two or more of the above components. The indirect communications may occur with aid of one or more intermediary device or network. For instance, indirect communications may utilize a telecommunications network. Indirect communications may be performed with aid of one or more router, communication tower, satellite, or any other intermediary device or network. Examples of types of communications may include, but are not limited to: communications via the Internet, Local Area Networks (LANs), Wide Area Networks (WANs), Bluetooth®, Near Field Communication (NFC) technologies, networks based on mobile data protocols such as General Packet Radio Services (GPRS), GSM, Enhanced Data GSM Environment (EDGE), 3G, 4G, or Long Term Evolution (LTE) protocols, Infra-Red (IR) communication technologies, and/or Wi-Fi, and may be wireless, wired, or a combination thereof. In some embodiments, the network may be implemented using cell and/or pager networks, satellite, licensed radio, or a combination of licensed and unlicensed radio. The network may be wireless, wired, or a combination thereof.

One or more diagnostic test devices, mobile devices and/or servers may be connected or interconnected to one or more databases. The databases may be one or more memory devices configured to store data. Additionally, the databases may also, in some embodiments, be implemented as a computer system with a storage device. In one aspect, the databases may be used by components of the network layout to perform one or more operations consistent with the disclosed embodiments.

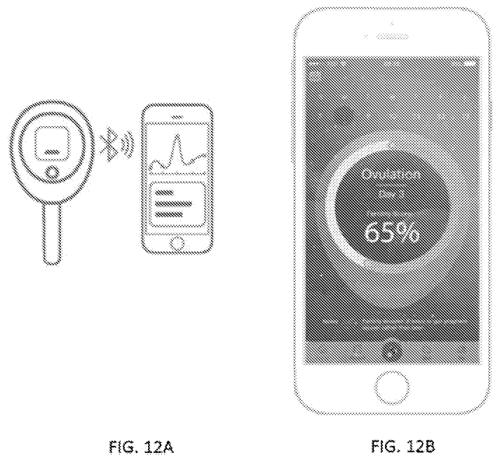

In some embodiments, one or more graphical user interfaces (GUIs) may be provided on the mobile device. The GUIs may be rendered on a display screen on the mobile device. A GUI is a type of interface that allows users to interact with electronic devices through graphical icons and visual indicators such as secondary notation, as opposed to text-based interfaces, typed command labels or text navigation. The actions in a GUI are usually performed through direct manipulation of the graphical elements. In addition to computers, GUIs can be found in hand-held devices such as MP3 players, portable media players, gaming devices and smaller household, office and industry equipment. The GUIs may be provided in a software, a software application, a web browser, etc. The GUIs may be displayed on the mobile device (e.g., FIG. 12B). The GUIs may be provided through a mobile application. The GUIs may be rendered through an application (e.g., via an application programming interface (API) executed on the mobile device). The GUIs may show images that permit a user to monitor fertility changes and levels.

As depicted in FIG. 12, the diagnostic test system may further comprise means for transmitting data generated by the diagnostic test system. In some cases, the data may be transmitted to and/or read from a mobile device (e.g., a cell phone, a tablet), a computer, a cloud application or any combination thereof. The data may be transmitted by any means for transmitting data, including, but not limited to, downloading the data from the system (e.g., USB, RS-232 serial, or other industry standard communications protocol) and wireless transmission (e.g., Bluetooth®, ANT+, NFC, or other similar industry standard). The information may be displayed as a report. The report may be displayed on the screen of a mobile device or a computer. The report may be transmitted to a healthcare provider or a caregiver. In some instances, the data may be downloaded to an electronic health record. Optionally, the data may comprise or be part of an electronic health record. For example, the data may be uploaded to an electronic health record of a user of the devices and methods described herein. In some cases, the data may be transmitted to a mobile device and displayed for a user on a mobile application, as shown in FIG. 12B.

Data collected by and transmitted by the diagnostic test system may include results of the immunoassay test performed on the immunoassay test device. For example, the data may include the concentrations of analytes (such as a first analyte and a second analyte) present in a sample. The concentration may relative concentrations or absolute concentrations. Data may also include an outcome such as a diagnostic outcome or a prognostic outcome, as shown in FIG. 12B.

Additional data that may be transmitted by the diagnostic test system include, without limitation, patient information, information obtained from the readable chip of the immunoassay device, time and date of the tests, system status (testing temperature, battery status, system self-testing and calibration results), error codes or error messages.

Though adapted for use with a fluid biological sample, the test wand may be used with solid biological matter (e.g., fecal matter) by modifying the solid matter to a fluid form, for example, by dissolving or disrupting (e.g., lysing) the solid matter in a liquid medium. Molecules contained in cell membranes and/or cell walls may also be released into a liquid medium in such cases. A liquid medium may include water, saline, cell-culture medium, or any solution and may contain any number of salts, surfactants, buffers, reducing agents, denaturants, preservatives, and the like.

Non-limiting examples of biological samples suitable for use with test wands of the disclosure include: whole blood, blood serum, blood plasma, urine, feces, saliva, vaginal secretions, semen, interstitial fluid, mucus, sebum, sweat, tears, crevicular fluid, aqueous humour, vitreous humour, bile, breast milk, cerebrospinal fluid, cerumen, enolymph, perilymph, gastric juice, peritoneal fluid, vomit, and the like. The biological sample may be obtained from a hospital, laboratory, clinical or medical laboratory. In some cases, the immunoassay test is performed by a clinician or laboratory technician. In other cases, the immunoassay test is performed by the subject, for example, at home.

The biological sample may be isolated initially from a multi-cellular organism in any suitable form, including though not limited to a human embryo or fetus. In some cases, the sample may be derived from a single or individual cell from a subject. In some cases, the sample may be an individual micro-organism, or a population of micro-organisms, or a mixture of micro-organisms and host cells.

In some cases, the biological sample comprises one or more bacterial cells. In some cases, the one or more bacterial cells are pathogens. In some cases, the one or more bacterial cells are infectious. Non-limiting examples of bacterial pathogens that can be detected include Mycobacteria (e.g. M. tuberculosis, M. bovis, M. avium, M. leprae, and M. africanum), Rickettsia, Mycoplasma, Chlamydia, and Legionella. Some examples of bacterial infections include, but are not limited to, infections caused by Gram positive Bacillus (e.g., Listeria, Bacillus such as Bacillus anthracis, Erysipelothrix species), Gram negative Bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, and Neisseria species. Specific examples of infectious bacteria include, but are not limited to: Helicobacter pyloris, Legionella pneumophilia, Mycobacterium tuberculosis, Mycobacterium avium, Mycobacterium intracellulare, Mycobacterium kansaii, Mycobacterium gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, Erysipelothrix rhusiopathiae, Clostridium tetani, Enterobacter aerogenes, Klebsiella pneumoniae, Pasturella multocida, Fusobacterium nucleatum, Streptobacillus moniliformis, Treponema pallidium, Treponema pertenue, Leptospira, Rickettsia, and Actinomyces israelii, Acinetobacter, Bacillus, Bordetella, Borrelia, Brucella, Campylobacter, Chlamydia, Chlamydophila, Clostridium, Corynebacterium, Enterococcus, Haemophilus, Helicobacter, Mycobacterium, Mycoplasma, Stenotrophomonas, Treponema, Vibrio, Yersinia, Acinetobacter baumanii, Bordetella pertussis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Corynebacterium diphtheriae, Enterobacter sazakii, Enterobacter agglomerans, Enterobacter cloacae, Enterococcus faecalis, Enterococcus faecium, Escherichia coli, Francisella tularensis, Helicobacter pylori, Legionella pneumophila, Leptospira interrogans, Mycobacterium leprae, Mycobacterium tuberculosis, Mycobacterium ulcerans, Mycoplasma pneumoniae, Pseudomonas aeruginosa, Rickettsia rickettsii, Salmonella typhi, Salmonella typhimurium, Salmonella enterica, Shigella sonnei, Staphylococcus epidermidis, Staphylococcus saprophyticus, Stenotrophomonas maltophilia, Vibrio cholerae, Yersinia pestis, and the like.

The biological sample may comprise one or more viruses. Non-limiting examples of viruses include the herpes virus (e.g., human cytomegalomous virus (HCMV), herpes simplex virus 1 (HSV-1), herpes simplex virus 2 (HSV-2), varicella zoster virus (VZV), Epstein-Barr virus), influenza A virus and Hepatitis C virus (HCV) or a picornavirus such as Coxsackievirus B3 (CVB3). Other viruses may include, but are not limited to, the hepatitis B virus, HIV, poxvirus, hepadavirus, retrovirus, and RNA viruses such as flavivirus, togavirus, coronavirus, Hepatitis D virus, orthomyxovirus, paramyxovirus, rhabdovirus, bunyavirus, filo virus, Adenovirus, Human herpesvirus, type 8, Human papillomavirus, BK virus, JC virus, Smallpox, Hepatitis B virus, Human bocavirus, Parvovirus B19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, Rubella virus, Hepatitis E virus, and Human immunodeficiency virus (HIV). In some cases, the virus is an enveloped virus. Examples include, but are not limited to, viruses that are members of the hepadnavirus family, herpesvirus family, iridovirus family, poxvirus family, flavivirus family, togavirus family, retrovirus family, coronavirus family, filovirus family, rhabdovirus family, bunyavirus family, orthomyxovirus family, paramyxovirus family, and arenavirus family. Other examples include, but are not limited to, Hepadnavirus hepatitis B virus (HBV), woodchuck hepatitis virus, ground squirrel (Hepadnaviridae) hepatitis virus, duck hepatitis B virus, heron hepatitis B virus, Herpesvirus herpes simplex virus (HSV) types 1 and 2, varicella-zoster virus, cytomegalovirus (CMV), human cytomegalovirus (HCMV), mouse cytomegalovirus (MCMV), guinea pig cytomegalovirus (GPCMV), Epstein-Barr virus (EBV), human herpes virus 6 (HHV variants A and B), human herpes virus 7 (HHV-7), human herpes virus 8 (HHV-8), Kaposi's sarcoma-associated herpes virus (KSHV), B virus Poxvirus vaccinia virus, variola virus, smallpox virus, monkeypox virus, cowpox virus, camelpox virus, ectromelia virus, mousepox virus, rabbitpox viruses, raccoonpox viruses, molluscum contagiosum virus, orf virus, milker's nodes virus, bovin papullar stomatitis virus, sheeppox virus, goatpox virus, lumpy skin disease virus, fowlpox virus, canarypox virus, pigeonpox virus, sparrowpox virus, myxoma virus, hare fibroma virus, rabbit fibroma virus, squirrel fibroma viruses, swinepox virus, tanapox virus, Yabapox virus, Flavivirus dengue virus, hepatitis C virus (HCV), GB hepatitis viruses (GBV-A, GBV-B and GBV-C), West Nile virus, yellow fever virus, St. Louis encephalitis virus, Japanese encephalitis virus, Powassan virus, tick-borne encephalitis virus, Kyasanur Forest disease virus, Togavirus, Venezuelan equine encephalitis (VEE) virus, chikungunya virus, Ross River virus, Mayaro virus, Sindbis virus, rubella virus, Retrovirus human immunodeficiency virus (HIV) types 1 and 2, human T cell leukemia virus (HTLV) types 1, 2, and 5, mouse mammary tumor virus (MMTV), Rous sarcoma virus (RSV), lentiviruses, Coronavirus, severe acute respiratory syndrome (SARS) virus, Filovirus Ebola virus, Marburg virus, Metapneumoviruses (MPV) such as human metapneumovirus (HMPV), Rhabdovirus rabies virus, vesicular stomatitis virus, Bunyavirus, Crimean-Congo hemorrhagic fever virus, Rift Valley fever virus, La Crosse virus, Hantaan virus, Orthomyxovirus, influenza virus (types A, B, and C), Paramyxovirus, parainfluenza virus (PIV types 1, 2 and 3), respiratory syncytial virus (types A and B), measles virus, mumps virus, Arenavirus, lymphocytic choriomeningitis virus, Junin virus, Machupo virus, Guanarito virus, Lassa virus, Ampari virus, Flexal virus, Ippy virus, Mobala virus, Mopeia virus, Latino virus, Parana virus, Pichinde virus, Punta toro virus (PTV), Tacaribe virus and Tamiami virus. In some embodiments, the virus is a non-enveloped virus, examples of which include, but are not limited to, viruses that are members of the parvovirus family, circovirus family, polyoma virus family, papillomavirus family, adenovirus family, iridovirus family, reovirus family, birnavirus family, calicivirus family, and picornavirus family. Specific examples include, but are not limited to, canine parvovirus, parvovirus B19, porcine circovirus type 1 and 2, BFDV (Beak and Feather Disease virus, chicken anaemia virus, Polyomavirus, simian virus 40 (SV40), JC virus, BK virus, Budgerigar fledgling disease virus, human papillomavirus, bovine papillomavirus (BPV) type 1, cotton tail rabbit papillomavirus, human adenovirus (HAdV-A, HAdV-B, HAdV-C, HAdV-D, HAdV-E, and HAdV-F), fowl adenovirus A, bovine adenovirus D, frog adenovirus, Reovirus, human orbivirus, human coltivirus, mammalian orthoreovirus, bluetongue virus, rotavirus A, rotaviruses (groups B to G), Colorado tick fever virus, aquareovirus A, cypovirus 1, Fiji disease virus, rice dwarf virus, rice ragged stunt virus, idnoreovirus 1, mycoreovirus 1, Birnavirus, bursal disease virus, pancreatic necrosis virus, Calicivirus, swine vesicular exanthema virus, rabbit hemorrhagic disease virus, Norwalk virus, Sapporo virus, Picornavirus, human polioviruses (1-3), human coxsackieviruses A1-22, 24 (CA1-22 and CA24, CA23 (echovirus 9)), human coxsackieviruses (B1-6 (CB1-6)), human echoviruses 1-7, 9, 11-27, 29-33, vilyuish virus, simian enteroviruses 1-18 (SEV1-18), porcine enteroviruses 1-11 (PEV1-11), bovine enteroviruses 1-2 (BEV1-2), hepatitis A virus, rhinoviruses, hepatoviruses, cardio viruses, aphthoviruses and echoviruses. The virus may be phage. Examples of phages include, but are not limited to T4, T5, λ phage, T7 phage, G4, P1, φ6, Thermoproteus tenax virus 1, M13, MS2, Qβ, φX174, Φ29, PZA, Φ15, BS32, B103, M2Y (M2), Nf, GA-1, FWLBc1, FWLBc2, FWLLm3, B4. In some cases, the virus is selected from a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus. In some cases, the virus is selected from a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Whitewater Arroyo virus, Chapare virus, and Lujo virus. In some cases, the virus is selected from a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, Punta Toro virus (PTV), California encephalitis virus, and Crimean-Congo hemorrhagic fever (CCHF) virus. In some cases, the virus is selected from a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus; a member of the Poxyiridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Ban virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1N1 swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T-lymphotropic virus Types I and II (HTLV-1 and HTLV-2, respectively).

The biological sample may comprise one or more fungi. Examples of infectious fungal agents include, without limitation Aspergillus, Blastomyces, Coccidioides, Cryptococcus, Histoplasma, Paracoccidioides, Sporothrix, and at least three genera of Zygomycetes. The above fungi, as well as many other fungi, can cause disease in pets and companion animals. The present teaching is inclusive of substrates that contact animals directly or indirectly. Examples of organisms that cause disease in animals include Malassezia furfur, Epidermophyton floccosur, Trichophyton mentagrophytes, Trichophyton rubrum, Trichophyton tonsurans, Trichophyton equinum, Dermatophilus congolensis, Microsporum canis, Microsporu audouinii, Microsporum gypseum, Malassezia ovale, Pseudallescheria, Scopulariopsis, Scedosporium, and Candida albicans. Further examples of fungal infectious agent include, but are not limited to, Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus.

The biological sample may comprise one or more parasites. Non-limiting examples of parasites include Plasmodium, Leishmania, Babesia, Treponema, Bonelia, Trypanosoma, Toxoplasma gondii, Plasmodium falciparum, P. vivax, P. ovale, P. malariae, Trypanosoma spp., or Legionella spp. In some cases, the parasite is Trichomonas vaginalis.

In some cases, the biological sample is a sample taken from a subject infected with or suspected of being infected with an infectious agent (e.g., bacteria, virus). In some aspects, the biological sample comprises an infectious agent associated with a sexually-transmitted disease (STD) or a sexually-transmitted infection (STI). Non-limiting examples of STDs or STIs and associated infectious agents that may be detected with the devices and methods provided herein may include, Bacterial Vaginosis; Chlamydia (Chlamydia trachomatis); Genital herpes (herpes virus); Gonorrhea (Neisseria gonorrhoeae); Hepatitis B (Hepatitis B virus); Hepatitis C (Hepatitis C virus); Genital Warts, Anal Warts, Cervical Cancer (Human Papillomavirus); Lymphogranuloma venereum (Chlamydia trachomatis); Syphilis (Treponema pallidum); Trichomoniasis (Trichomonas vaginalis); Yeast infection (Candida); and Acquired Immunodeficiency Syndrome (Human Immunodeficiency Virus).

The sample can be from an environmental source or an industrial source. Examples of environmental sources include, but are not limited to, agricultural fields, lakes, rivers, water reservoirs, air vents, walls, roofs, soil samples, plants, and swimming pools. Examples of industrial sources include, but are not limited to clean rooms, hospitals, food processing areas, food production areas, food stuffs, medical laboratories, pharmacies, and pharmaceutical compounding centers. The sample can be a forensic sample (e.g., hair, blood, semen, saliva, etc.). The sample can comprise an agent used in a bioterrorist attack (e.g., influenza, anthrax, smallpox).

The biological sample can be from a subject (e.g., human subject) who is healthy. The biological sample can be from a pregnant female mammal. In some cases, the biological sample is taken from the pregnant female mammal at any time from about 4 to about 26 weeks of gestation. The biological sample may be taken from a female mammal during a menstrual cycle. In some cases, the biological sample is taken from a female mammal at any time from about 1 to about 32 days from the last menstrual period. The biological sample may be taken from a female mammal during an ovulation cycle. In some cases, the biological sample is taken from an ovulating female mammal at any time from about 11 to about 21 days from the last menstrual period. The biological sample may be taken from a female mammal to determine a time of elevated fertility, for example, a period of time during the ovulation cycle in which the female mammal is most likely to become pregnant.

In some cases, more than one sample can be obtained from a subject or source and multiple immunoassay tests (e.g., utilizing multiple immunoassay devices) can be performed. In some cases, anywhere from 2 to 50 or more samples may be obtained. In some cases, more than one sample may be obtained over a period of time, for example, to monitor disease progression or to monitor a biological state or condition (e.g., fertility status). Generally, test wands of the disclosure are configured for one-time use (e.g., disposable).

In some cases, the subject is affected by a genetic disease, a carrier for a genetic disease or at risk for developing or passing down a genetic disease, where a genetic disease is any disease that can be linked to a genetic variation such as mutations, insertions, additions, deletions, translocation, point mutation, trinucleotide repeat disorders and/or single nucleotide polymorphisms (SNPs).

The biological sample can be from a subject who has a specific disease, disorder, or condition, or is suspected of having (or at risk of having) a specific disease, disorder or condition. For example, the biological sample can be from a cancer patient, a patient suspected of having cancer, or a patient at risk of having cancer. The cancer can be, e.g., acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloeptithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, nonmelanoma, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom Macroglobulinemia, or Wilms Tumor. The sample can be from the cancer and/or normal tissue from the cancer patient. In some cases, the sample is a biopsy of a tumor.

The test wands may be provided with an immunoassay configured to detect the presence of anywhere from 1 to 10 or more analytes in a sample. The device may be used to test for the presence or absence of at least a first analyte and a second analyte in a sample. In some cases, the device may be used to determine an amount or a relative amount of at least a first and second analyte in a sample.

The presence or absence of analytes may be indicative of a disease or disorder in a subject. The presence or absence of analytes may be indicative of a biological state or condition of a subject. In one example, the presence or absence of analytes may indicate the fertility of a female mammal. The presence or absence of analytes may indicate that a female mammal has an elevated fertility or is at a particular period of time in an ovulation cycle. In some cases, the presence or absence of analytes indicates that a female mammal is pregnant. In some cases, the presence or absence of analytes indicates the gestation time of a female mammal. In some cases, the presence or absence of analytes indicates that a subject has or is at risk of developing a disease. In some cases, the presence or absence of analytes indicates that a subject has a disorder (e.g., thyroid disorder). In some cases, the presence or absence of analytes indicates that a subject has a deficiency (e.g., vitamin deficiency). In some cases, the presence or absence of analytes indicates that a product (e.g., a food or drink product) contains an allergen.

Reagents may include an antibody or antibody fragment, an antigen, an aptamer, a peptide, a small molecule, a ligand, a molecular complex or any combination thereof. A reagent may be any reagent that has specific binding activity for a respective analyte. In particular cases, the reagent may be antibodies or antibody fragments that specifically bind to epitopes present on the analyte. The immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule. In some cases, the antibody is an antigen-binding antibody fragment such as, for example, a Fab, a F(ab′), a F(ab′)2, a Fd chain, a single-chain Fv (scFv), a single-chain antibody, a disulfide-linked Fv (sdFv), a fragment comprising either a VL or VH domain, or fragments produced by a Fab expression library. Antigen-binding antibody fragments, including single-chain antibodies, can comprise the variable region(s) alone or in combination with the entirety or a portion of the following: hinge region, CH1, CH2, CH3 and CL domains. Also, antigen-binding fragments can comprise any combination of variable region(s) with a hinge region, CH1, CH2, CH3 and CL domains. Antibodies and antibody fragments may be derived from a human, rodent (e.g., mouse and rat), donkey, sheep, rabbit, goat, guinea pig, camelid, horse, or chicken. Various antibodies and antibody fragments may be designed to selectively bind essentially any desired analyte. Methods of generating antibodies and antibody fragments are well known in the art.

The terms “selective” or “specific” binding may be used herein interchangeably. Generally speaking, a ligand that selectively or specifically binds to a target means that the ligand has a high binding affinity for its target, and a low binding affinity for non-target molecules. The dissociation constant (Kd) may be used herein to describe the binding affinity of a ligand for a target molecule (e.g., an analyte). The dissociation constant may be defined as the molar concentration at which half of the binding sites of a target molecule are occupied by the ligand. Therefore, the smaller the Kd, the tighter the binding of the ligand to the target molecule. In some cases, a ligand has a dissociation constant (Kd) for a target molecule of less than 1 mM, less than 100 μM, less than 10 μM, less than 1 μM, less than 100 nM, less than 50 nM, less than 25 nM, less than 10 nM, less than 5 nM, less than 1 nM, less than 500 pM, less than 100 pM, less than 50 pM, or less than 5 pM.

Methods of retaining reagents to a test strip are widely known in the art and essentially any method may be used. A detection reagent may be retained in mobilizable manner such that in a wet state, for example, when a liquid saturates the labeling zone retaining the detection reagent, the detection reagent becomes freely mobile along the flow path. Capture and control reagents may be retained in an immobilized manner such that they remain immobile on the flow path even when a liquid saturates the capture and control regions. Non-limiting examples for retaining a reagent in an immobilized manner include direct adsorption to a membrane and covalent attaching of the reagent to a linker molecule.

Further provided herein are kits which may include any number of immunoassay test devices and/or reader devices of the disclosure. In one aspect, a kit is provided for determining qualitatively or quantitatively the presence of at least a first analyte and a second analyte in a biological sample, the kit comprising: a) an assay device according to an embodiment of the disclosure; and b) instructions for using the kit.

In some cases, kits may include a one or more immunoassay test devices of the disclosure. In some cases, the kit may provide a plurality of immunoassay devices to enable a user to conduct a test on more than one occasion. In some cases, the immunoassay devices are configured for a single use (i.e., are disposable). A kit may include a plurality of test devices to enable a user to perform a test once a day, once every 2 days, once every 3 days, once every 4 days, once every 5 days, once every 6 days, once every week, once every 2 weeks, once every 3 weeks, once every 4 weeks, once every 5 weeks, once every 6 weeks once every 7 weeks, once every 8 weeks or more.

In some cases, kits may include a plurality of immunoassay devices, each capable of detecting the same analytes. In other cases, kits may include a plurality of immunoassay devices, each capable of detecting different analytes. In a particular embodiment, a kit may include a plurality of immunoassay devices, each of the immunoassay devices capable of detecting the presence of E3G and LH in a biological sample. In another particular embodiment, a kit may include a plurality of immunoassay devices, each of the immunoassay devices capable of detecting the presence of LH in a biological sample. In another particular embodiment, a kit may include a plurality of immunoas say devices, each of the immunoassay devices capable of detecting the presence of hCG in a biological sample. In another particular embodiment, a kit may include a plurality of immunoassay devices, each of the immunoassay devices capable of detecting the presence of Influenza A & B in a biological sample. In another particular embodiment, a kit may include a plurality of immunoassay devices, each of the immunoassay devices capable of detecting the presence of 25-hydroxyvitamin D in a biological sample. In another particular embodiment, a kit may include a plurality of immunoassay devices, each of the immunoassay devices capable of detecting the presence of triiodothyronine (T3) and thyroxine (T4) in a biological sample. In another particular embodiment, a kit may include a plurality of immunoassay devices, each of the immunoassay devices capable of detecting the presence of one or more allergens in a food or drink product.

In some cases, kits can be provided with instructions. The instructions can be provided in the kit or they can be accessed electronically (e.g., on the World Wide Web). The instructions can provide information on how to use the devices and/or systems of the present disclosure. The instructions can provide information on how to perform the methods of the disclosure. In some cases, the kit can be purchased by a physician or health care provider for administration at a clinic or hospital. In other cases, the kit can be purchased by the subject and self-administered (e.g., at home). In some cases, the kit can be purchased by a laboratory.

Kits may further comprise a diagnostic test system of the disclosure. The diagnostic test system may be configured to be used with the immunoassay test devices of the disclosure. In some cases, the diagnostic test system is configured to be in operable communication with the assay device.

In one aspect, a method is provided for detecting the presence of a first analyte and a second analyte in a biological sample. The method may comprise: a) contacting a first end of a test strip with a biological sample suspected of containing the first analyte and the second analyte. The method may further comprise: b) reacting the biological sample at a labeling zone with a mobilizable first detection reagent conjugated to a first fluorescent label and a mobilizable second detection reagent conjugated to a second fluorescent label, which first detection reagent specifically binds to the first analyte thereby forming a first analyte-first detection reagent complex and the second detection reagent specifically binds to the second analyte thereby forming a second analyte-second detection reagent complex. The method may further comprise: c) flowing the biological sample from the labeling zone to a capture zone comprising a first capture region and a second capture region, wherein the first capture region comprises a first capture reagent immobilized thereon which specifically binds to the first detection reagent when the first detection reagent is not in a complex with the first analyte, and wherein the second capture region comprises a second capture reagent immobilized thereon which specifically binds to the second detection reagent-second analyte complex. The method may further comprise: d) detecting: (i) a first optical signal from the first fluorescent label present at the first capture region which first optical signal decreases with increasing amounts of the first analyte present in the biological sample; and (ii) a second optical signal from the second fluorescent label present at the second capture region which second optical signal increases with increasing amounts of the second analyte present in the biological sample, thereby detecting a presence of the first analyte and the second analyte present in the biological sample.

In various aspects, the methods may involve the use of one or more immunoassay test devices provided by the disclosure. The methods generally involve contacting the test device comprising a test strip with a sample by, for example, contacting a sample zone of the device with a fluid sample. Examples of samples suitable for use with the test devices have been described. The sample may be flowed along a flow path of the test strip from a proximal end to the distal end of the test strip. In some cases, the sample is flowed by capillarity or wicking.

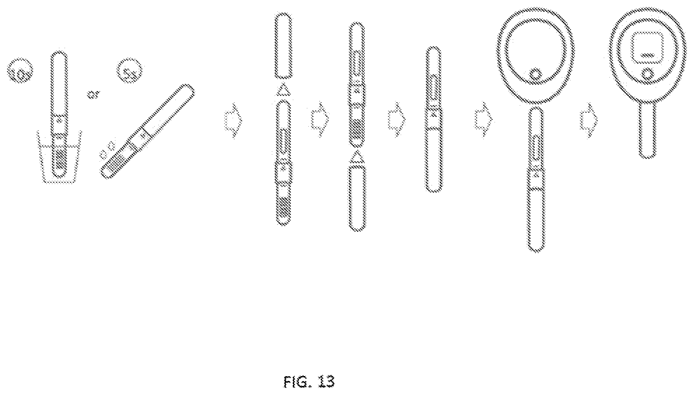

FIG. 13 depicts an example workflow of a method of using an immunoassay test device to perform an immunoassay, and then obtaining a result using a diagnostic test system. An immunoassay test device is contacted with a fluid sample, either by inserting the test device into a container holding a fluid sample for a period of time (e.g., 10 seconds) or by holding the test device under a fluid stream for a period of time (e.g., 5 seconds). A cap may be removed from the distal end of the immunoassay device to reveal the assay results window and another cap may be inserted over the proximal end of the immunoassay device to cover and protect the sample zone. After waiting for a period of time for the immunoassay to be completed, the immunoassay test device may be inserted into the receiving chamber of a diagnostic test system, and the diagnostic test system may scan the immunoassay test device to obtain a result.

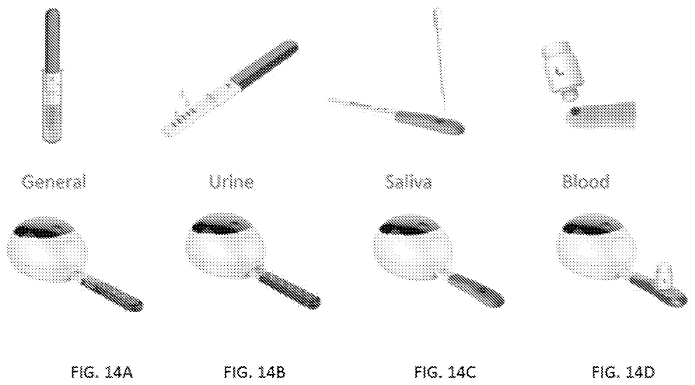

FIG. 14 depicts methods of applying various biological samples to a sample zone of an immunoassay device. Biological samples may include urine, saliva, and blood, among others as described herein. The fluid sample may be applied to a sample zone of an immunoassay test device by, e.g. inserting the proximal end of the device into a contained holding the sample, by holding the proximal end of the device under a fluid stream (e.g., a urine stream), by pipetting a fluid sample onto the sample zone of the device, or by using a needle or other pricking device to obtain a blood sample.

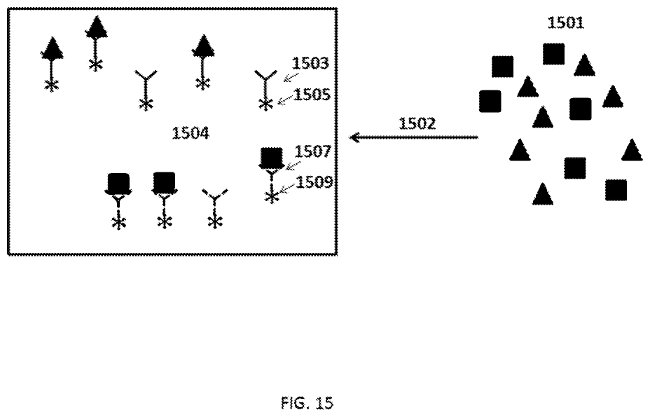

FIG. 15 depicts a non-limiting example of a labeling reaction as performed on a test device of the disclosure. A sample 1501 suspected of containing a first analyte (triangles) and a second analyte (squares) may be flowed 1502 from a sample zone to a labeling zone 1504 containing a mobilizable first detection reagent 1503 conjugated to a first detectable label 1505 and a mobilizable second detection reagent 1507 conjugated to a second detectable label 1509. The first detection reagent 1503 may be capable of selectively binding to the first analyte (triangles), when present in the sample, and the second detection reagent 1507 may be capable of selectively binding to the second analyte (squares), when present in the sample.

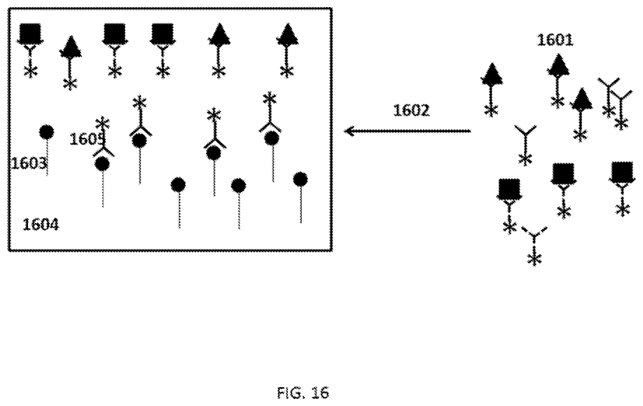

FIG. 16 depicts a non-limiting example of a competitive binding-based assay as performed on a test device of the disclosure. After a first analyte and second analyte, when present in the sample, have been labeled (see FIG. 15), the labeled sample 1601 may be flowed 1602 along the test strip from the labeling zone to a capture zone comprising a first capture region 1604. The first capture region 1604 may have immobilized thereon a first capture reagent 1603. The first capture reagent 1603 may be capable of specifically binding to non-complexed first detection reagent 1605 (i.e., when the first detection reagent is not in a complex with the first analyte). The first capture reagent 1603 may not be capable of binding to the first analyte or the first analyte-first detection reagent complex.

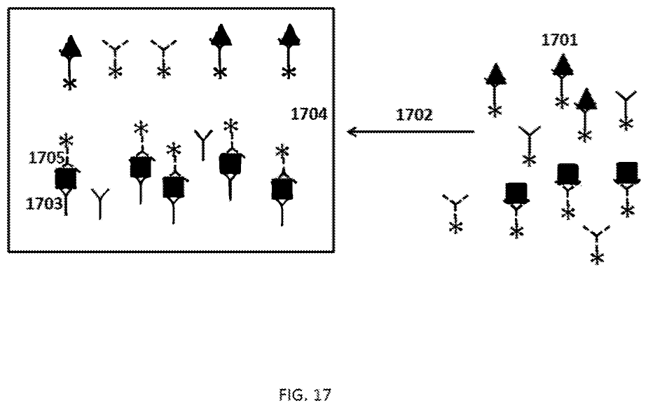

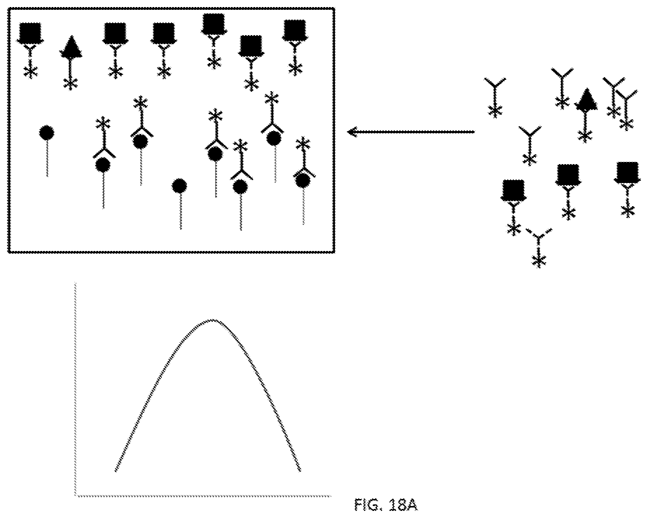

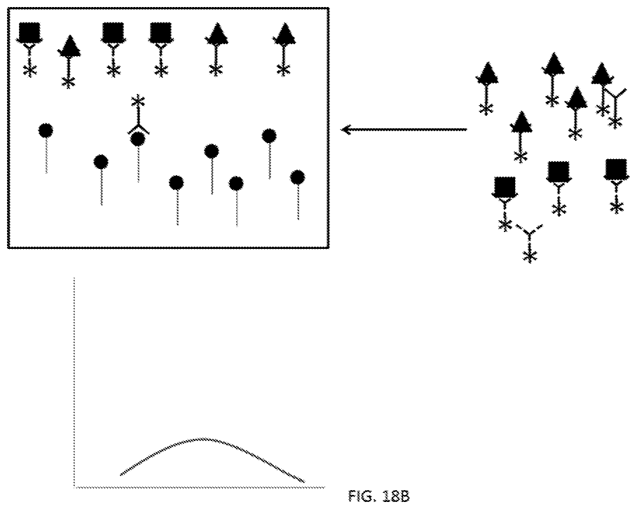

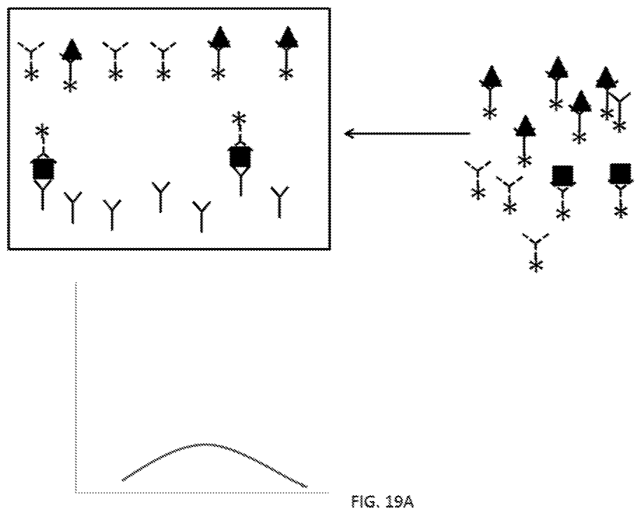

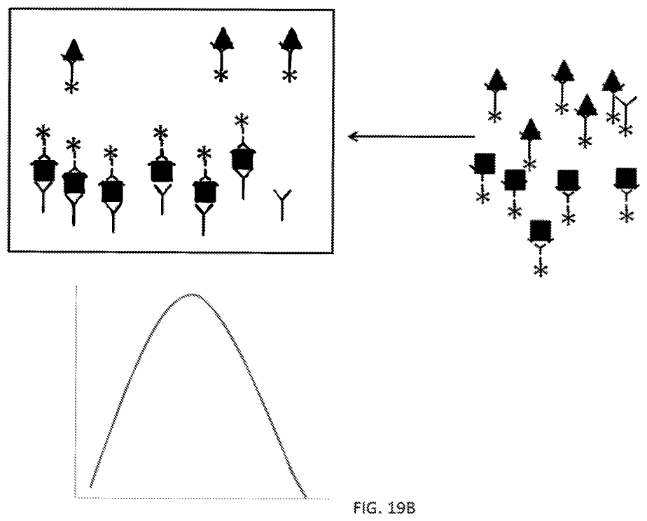

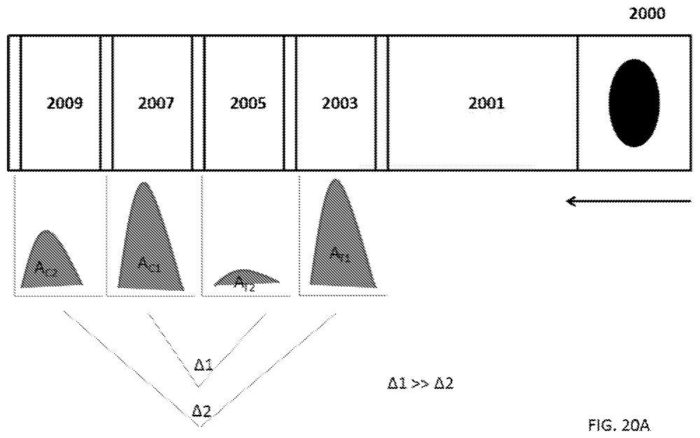

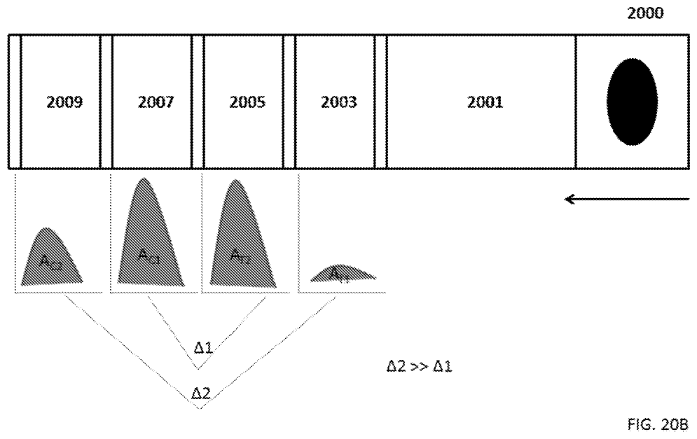

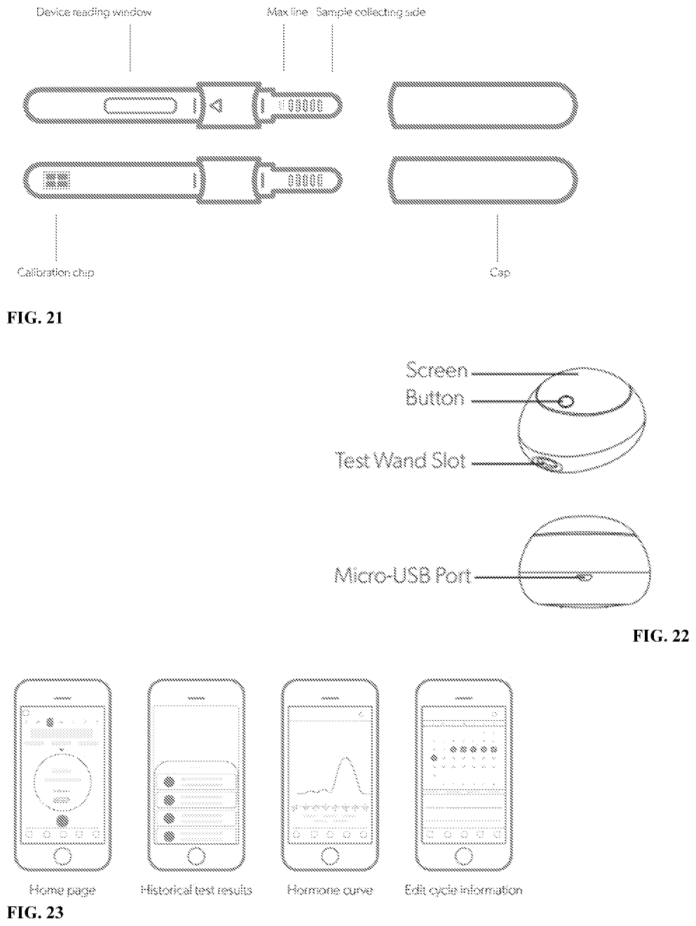

FIG. 17 depicts a non-limiting example of a sandwich-based assay as performed on a test device of the disclosure. After a first analyte and second analyte, when present in the sample, have been labeled (see FIG. 15), the labeled sample 1701 may be flowed 1702 along the test strip from the labeling zone to a capture zone comprising a second capture region 1704. The second capture region 1704 may have immobilized thereon a second capture reagent 1703. The second capture reagent may be capable of specifically binding to second analyte-second detection reagent complex 1705, for example, as formed by the labeling reaction in FIG. 15. The second capture reagent may not be capable of binding to the second analyte alone or the second detection reagent alone.

FIG. 18A depicts a non-limiting example of utilizing a competitive binding-based assay as performed on a test device of the disclosure to detect the absence of (or low levels of) a first analyte in the sample. A labeled sample (see FIG. 15) may be flowed from the labeling zone to a capture zone comprising a first capture region, as shown in FIG. 16. When the levels of first analyte in the sample are low, or absent, less first analyte-first detection reagent complex is formed at the labeling zone, and therefore, more free first detection reagent is available for binding to the first capture reagent immobilized at the first capture region. Thus, the signal produced by measuring the amount of first detectable label at the first capture region will be high. In contrast, FIG. 18B depicts a non-limiting example of utilizing a competitive binding-based assay as performed on a test device of the disclosure to detect high levels of first analyte present in the sample. When the levels of first analyte in the sample are high, more first analyte-first detection reagent complex is formed at the labeling zone, and therefore, less free first detection reagent is available for binding to the first capture reagent immobilized at the first capture region. Thus, the signal produced by measuring the amount of first detectable label at the first capture region will be low, or absent.

FIG. 19A depicts a non-limiting example of utilizing a sandwich-based assay as performed on a test device of the disclosure to detect the absence of (or low levels of) second analyte in the sample. A labeled sample (see FIG. 15) may be flowed from the labeling zone to a capture zone comprising a second capture region, as shown in FIG. 16. When the levels of second analyte in the sample are low, or absent, less second analyte-second detection reagent is formed at the labeling zone, and therefore, less second analyte-second detection reagent is available for binding to the second capture reagent immobilized at the second capture region. Thus, the signal produced by measuring the amount of second detectable label at the second capture region will be low. In contrast, FIG. 19B depicts a non-limiting example of utilizing a sandwich-based assay as performed on a test device of the disclosure to detect high levels of second analyte present in the sample. When the levels of second analyte in the sample are high, more second analyte-second detection reagent complex is formed at the labeling zone, and therefore, more second analyte-second detection reagent complex is available for binding to the second capture reagent immobilized at the second capture region. Thus, the signal produced by measuring the amount of second detectable label at the second capture region will be low, or absent.

FIGS. 20A & 20B demonstrate the detection of a first analyte and a second analyte present in a sample by utilizing an immunoassay device of the disclosure. FIG. 20A demonstrates an example of a negative test result (e.g., when a first analyte and a second analyte are absent from or present in low amounts in a sample). The amount of first analyte present in the sample may be detected at the first capture region and the amount of second analyte present in the sample may be detected at the second capture region, in accordance with the disclosure. When the amount of first analyte present in the sample is low or absent, the signal produced at the first capture region is high, whereas when the amount of second analyte present in the sample is low or absent, the signal produced at the second capture region is low. Further, the sample may be further flowed to a control zone comprising a first control region and a second control region. The first control region and second control region have immobilized thereon a first and second control reagent, respectively, capable of binding to excess first and second detection reagent present in the control zone. As shown in FIG. 20A, optical signal intensity may be measured at each position along the immunoassay device. The area under the curve may be calculated to determine the signal intensity for each discrete region of the device. The difference between the signal intensity at the first control region (AC1) and the signal intensity at the second capture region (AT2) may be determined (Δ1). Further, the difference between the signal intensity at the second control region (AC2) and the signal intensity at the first capture region (ATI) may be determined (Δ2). Δ1 and Δ2 may be compared and a negative result is outputted if Δ1 is greater than Δ2. Similarly, FIG. 20B demonstrates an example of a positive result (e.g., both the first analyte and second analyte are present in the sample in high amounts). When the amount of first analyte present in the sample is high, the signal intensity at the first capture region is low, whereas when the amount of second analyte present in the sample is low, the signal intensity at the second capture region is high. The difference between the signal intensity at the first control region (AC1) and the signal intensity at the second capture region (AT2) may be determined (Δ1). Further, the difference between the signal intensity at the second control region (AC2) and the signal intensity at the first capture region (AT1) may be determined (Δ2). Δ1 and Δ2 may be compared and a positive result is outputted if Δ1 is less than Δ2.

In some cases, the difference between the signal intensity of the first capture region and the signal intensity of the second capture region may be calculated. In some cases, the difference between the signal intensity of the first control region and the signal intensity of the second control region may be calculated. In some cases, the difference between the signal intensity of the first test region and the first control region may be calculated. In some cases, the difference between the signal intensity of the second test region and the second control region may be calculated.

The methods and devices herein may be used to detect the presence of one or more analytes in a sample. The methods and devices herein may be capable of detecting at least about 5 ng/ml analyte in a sample. For example, the methods and devices herein may be capable of detecting analyte in a sample in an amount from about 5 ng/ml to about 1000 ng/ml or greater. In one non-limiting example, a device is configured to detect the amounts of estrone-3-glucoronide (E3G) and luteinizing hormone (LH) present in a urine sample of a female mammal. Without wishing to be bound by theory, increased levels of E3G and LH in a urine sample may indicate that a female mammal is at a time of peak or elevated fertility, and thus, an increased chance of becoming pregnant. On the other hand, low levels of E3G and LH in a urine sample may indicate that a female mammal is at a time of low fertility, and thus, a decreased chance of becoming pregnant. The methods and devices described herein may exhibit increased sensitivity as compared to competing devices currently on the market.

In one non-limiting example, a device and methods of using the device in accordance with embodiments described herein are provided. A female subject wishes to know if she is currently at a time of low fertility or high fertility. The subject takes a urine sample by urinating into a container and then dips a device of the disclosure into the urine sample such that the sample zone of the device is contacted with the urine sample. The urine sample is suspected of containing E3G and LH. The urine sample is flowed from the sample zone to a labeling zone. The labeling zone comprises a mobilizable anti-E3G antibody conjugated to a first fluorophore, and a mobilizable anti-LH antibody conjugated to a second fluorophore. The anti-E3G antibody is capable of binding to E3G, when present in the urine sample, thereby forming a complex with E3G. Similarly, the anti-LH antibody is capable of binding to LH, when present in the urine sample, thereby forming a complex a LH. The complexes, if formed, as well as any non-complexed anti-E3G antibody and anti-LH antibody are flowed from the labeling zone to the capture zone. The capture zone comprises two capture regions: a first capture region and a second capture region. The first capture region and second capture region may be positioned in any order on the flow path of the device, for example, the first capture region may be downstream from the second capture region, or the first capture region may be upstream from the second capture region. The first capture region comprises an immobilized protein-E3G antigen complex which is capable of binding to the anti-E3G antibody, but not E3G or a complex of E3G. When the levels of E3G present in the urine sample are low or absent, less E3G are available to bind to the anti-E3G antibody at the labeling zone, and more non-complexed anti-E3G antibody are available to bind to the immobilized protein-E3G antigen complex at the first capture region. In contrast, when the levels of E3G present in the urine sample are high, more E3G are available to bind to the anti-E3G antibody at the labeling zone, and less non-complexed anti-E3G antibody are available to bind to the immobilized protein-E3G antigen complex at the first capture zone. The second capture region comprises an immobilized anti-LH antibody which is capable of binding to complexed LH and anti-LH antibody formed at the labeling zone, but not non-complexed LH or non-complexed anti-LH antibody. When the levels of LH present in the urine sample are low or absent, less LH is available to bind to the anti-LH antibody at the labeling zone thereby forming less anti-LH antibody-LH complexes, and less anti-LH antibody-LH complexes are available to bind to the anti-LH antibody present at the second capture region. In contrast, when the levels of LH present in the urine sample are high, more LH is available to bind to the anti-LH antibody at the labeling zone thereby forming more anti-LH antibody-LH complexes, and more anti-LH antibody-LH complexes are be available to bind to the anti-LH antibody present at the second capture region.

In one non-limiting embodiment, the sample is further flowed from the capture zone to a control zone. The control zone comprises a first control region and a second control region. The first control region comprises an anti-mouse IgG capable of binding to any first and second detection reagents that are present in the first control region. Likewise, the second control region comprises an anti-mouse IgG capable of binding to any first and second detection reagents that are present in the second control region.

After the test has been conducted on the immunoassay device, the device is inserted into a diagnostic test device of the disclosure. The diagnostic test device is configured to scan the immunoassay device and measure the levels of fluorescent label present at the first capture region and the second capture region, corresponding to the amount of E3G and LH present in the original sample, respectively. The diagnostic test device is further configured to measure the levels of fluorescent label present at the first control region and the second control region corresponding to the amount of excess first detection reagent and second detection reagent present in the control zone.

The amounts of E3G and LH present in the urine sample are calculated by integrating to find the area under the curve of the signal intensity plots. The measurement results (e.g., amounts of E3G and LH present in the urine sample) may be displayed on a display screen of the diagnostic test device. The measurement results may further be transmitted to a mobile application on a mobile device (e.g., via Bluetooth®), as depicted in FIG. 12B.

The mobile application may perform one or more algorithms on the measurement results. For example, the difference between the signal intensity of the first capture region corresponding to the amount of E3G present in the sample and the signal intensity of the second control region is calculated (Δ1). The difference between the signal intensity of the second capture region corresponding to the amount of LH present in the sample and the signal intensity of the first control region is calculated (Δ2). Δ1 is compared to Δ2 and if Δ1 is greater than Δ2, a negative result is returned. In contrast, if Δ2 is greater than Δ1, a positive result is returned. In some cases, the algorithm may analyze the measurement results based on the user's historic data. For example, the algorithm may calculate one or more of the following: a) E3G and LH base line calculation; b) Previous cycle peak/maximum values and date; c) E3G increase slope.

A result may be displayed on a GUI provided on a mobile device for a user to view. The result may indicate the day of ovulation (e.g., a time from the last menstrual period) and/or may indicate a fertility score.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Although the present invention is described with reference to particular embodiments, it will be understood to those skilled in the art that the foregoing disclosure addresses exemplary embodiments only; that the scope of the invention is not limited to the disclosed embodiments; and that the scope of the invention may encompass any combination of the disclosed embodiments, in whole or in part, as well as additional embodiments embracing various changes and modifications relative to the examples disclosed herein without departing from the scope of the invention as defined in the appended claims and equivalents thereto.

It will be understood that the disclosed invention, and components thereof, may be entirely manufactured and assembled prior to a first use of the invention by an intended end-user, or may be provided as a kit of individual components intended for assembly by a user prior to a first use.

To the extent necessary to understand or complete the disclosure of the present invention, all publications, patents, and patent applications mentioned herein are expressly incorporated by reference herein to the same extent as though each were individually so incorporated. No license, express or implied, is granted to any patent incorporated herein.

The present invention is not limited to the exemplary embodiments illustrated herein, but is instead characterized by the appended claims, which in no way limit the scope of the disclosure.

What is claimed is:

1. A method of detecting a first analyte in a biological sample, said method comprising:
(a) contacting a sampling zone of a test strip with said biological sample suspected of containing said first analyte;
(b) reacting said biological sample, at a first labeling zone of said test strip, with a mobilizable first detection reagent conjugated to a first fluorescent label, said first detection reagent being adapted to bind to said first analyte to form a first analyte-reagent complex, and said first fluorescent label being adapted to emit light upon excitation;
(c) irradiating said biological sample with light at a first wavelength to excite said first fluorescent label, said first fluorescent label emitting light at a second wavelength upon excitation;
(d) detecting said light emitted at said second wavelength from said first fluorescent label; and
(e) estimating a concentration of said first analyte based on an intensity of said detected light emitted at said second wavelength;
wherein steps (c)-(e) are performed with an analyzer comprising: a light emitting source for emitting said light at said first wavelength, a light detector for detecting said light emitted at said second wavelength, and a processor for estimating a concentration if said first analyte,
wherein said test strip comprises a storage device storing information pertaining to said first analyte to be detected with use of said test strip, and said analyzer comprises a reading device for reading stored information from said storage device, and
wherein said analyzer estimates a concentration of said first analyte based on predetermined information informing on a correlation between an emission character of said first fluorescent label and a concentration of said first analyte in said first analyte-reagent complex.

2. The method of claim 1, wherein
said predetermined information is stored in said storage device of said test strip, and said analyzer estimates a concentration of said first analyte based on said predetermined information upon reading said predetermined information from said storage device of said test strip.

3. The method of claim 1, wherein
said analyzer comprises predetermined information for several different correlations of an analytes and a fluorescent label, and said analyzer selects predetermined information for correlating said first analyte and said first fluorescent label based on information read from said storage device of said test strip.

4. The method of claim 1, wherein
said predetermined information is stored as a standard curve that informs a relationship between analyte concentration and fluorescent label intensity.

5. The method of claim 1, wherein
said light detector is adapted to distinguish between light emitted by said first fluorescent label at multiple discrete positions of said test strip, and said processor is configured to determine differences in intensity of said emitted light among said multiple discrete positions to obtain a scan pattern of said first fluorescent label.

6. The method of claim 5, wherein
said processor is configured to obtain said scanned pattern as a binary optical pattern.

7. The method of claim 5, wherein
said processor is configured to calculate one or more of a height, an area, and a size of a waveform of said emitted light based on said scan pattern.

8. The method of claim 1, further comprising:
(f) determining a status of a subject associated with said biological sample based on said estimated concentration of said first analyte.

9. The method of claim 8, wherein
said analyzer is configured to determine a status of said subject based on said estimated concentration of said first analyte in combination with pre-stored historical data comprising multiple prior estimated concentrations of said first analyte.

10. The method of claim 9, wherein
said analyzer is configured to determine a status of said subject by:
(i) establishing a baseline concentration of said first analyte based on an average of said prior estimated concentrations of said first analyte from said pre-stored historical data,
(ii) determining differential changes in concentration of said first analyte between consecutive estimated concentrations of said first analyte from said pre-stored historical data,
(iii) establish one or more dynamic thresholds for said first analyte based on said baseline concentration and said differential changes in concentration, and
(iv) determining if a current estimated concentration of said first analyte exceeds said one or more dynamic thresholds.

11. The method of claim 10, wherein
said analyzer is configured to determine a status of said subject without reliance on a predetermined fixed threshold value for an estimated concentration of said first analyte.

12. The method of claim 9, wherein
said pre-stored historical data is stored in a memory of said analyzer.

13. The method of claim 9, wherein
said analyzer is configured to communicate with a remote device for storage and/or display of said estimated concentration of said first analyte and/or said pre-stored historical data at said remote device.

14. The method of claim 8, wherein
said first analyte is a hormone or metabolite, and determining a status of the subject comprises determining a menstruation status.

15. The method of claim 9, wherein
said first analyte is a hormone or metabolite, and said pre-stored historical data comprises data informing hormonal changes of said subject during a menstrual cycle.

16. The method of claim 10, wherein
said first analyte is a hormone or metabolite, and said method further comprises:
(v) when it is determined that a current estimated concentration of said first analyte exceeds said one or more dynamic thresholds, a status of said subject is determined as that of ovulation.
